SEQUENCE LISTING (29 sequences)

SEQ ID NO: 1 - protein, 1196 aa, Herpes simplex virus
METKPKTATTIKVPPGPLGYVYARACPSEGIELLALLSARSGDADVAVAPLVVGLTVESGFEANVAVVVGSRTTGLGGTAVSLKLTPSHYSSSVYVFHGGRHLDPSTQAPNLTRLCERARRHFGFSDYTPRPGDLKHETTGEALCERLGLDPDRALLYLVVTEGFKEAVCINNTFLHLGGSDKVTIGGAEVHRIPVYPLQLFMPDFSRVIAEPFNANHRSIGENFTYPLPFFNRPLNRLLFEAVVGPAAVALRCRNVDAVARAAAHLAFDENHEGAALPADITFTAFEASQGKTPRGGRDGGGKGPAGGFEQRLASVMAGDAALALESIVSMAVFDEPPTDISAWPLCEGQDTAAARANAVGAYLARAAGLVGAMVFSTNSALHLTEVDDAGPADPKDHSKPSFYRFFLVPGTHVAANPQVDREGHVVPGFEGRPTAPLVGGTQEFAGEHLAMLCGFSPALLAKMLFYLERCDGGVIVGRQEMDVFRYVADSNQTDVPCNLCTFDTRHACVHTTLMRLRARHPKFASAARGAIGVFGTMNSMYSDCDVLGNYAAFSALKRADGSETARTIMQETYRAATERVMAELETLQYVDQAVPTAMGRLETIITNREALHTVVNNVRQVVDREVEQLMRNLVEGRNFKFRDGLGEANHAMSLTLDPYACGPCPLLQLLGRRSNLAVYQDLALSQCHGVFAGQSVEGRNFRNQFQPVLRRRVMDMFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAAIFPNGKPPGSNQPNPQWFWTALQRNQLPARLLSREDIETIAFIKKFSLDYGAINFINLAPNNVSELAMYYMANQILRYCDHSTYFINTLTAIIAGSRRPPSVQAAAAWSAQGGAGLEAGARALMDAVDAHPGAWTSMFASCNLLRPVMAARPMVVLGLSISKYYGMAGNDRVFQAGNWASLMGGKNACPLLIFDRTRKFVLACPRAGFVCAASNLGGGAHESSLCEQLRGIISEGGAAVASSVFVATVKSLGPRTQQLQIEDWLALLEDEYLSEEMMELTARALERGNGEWSTDAALEVAHEAEALVSQLGNAGEVFNFGDFGCEDDNATPFGGPGAPGPAFAGRKRAFHGDDPFGEGPPDKKGDLTLDML

SEQ ID NO: 2 - DNA, 3591 bp, Herpes simplex virus
atggagacaaagcccaagacggcaaccaccatcaaggtcccccccgggcccctgggatacgtgtacgctcgcgcgtgtccgtccgaaggcatcgagcttctggcgttactgtcggcgcgcagcggcgatgccgacgtcgccgtggcgcccctggtcgtgggcctgaccgtggagagcggctttgaggccaacgtagccgtggtcgtgggttctcgcacgacggggctcgggggtaccgcggtgtccctgaaactgacgccatcgcactacagctcgtccgtgtacgtctttcacggcggccggcacctggaccccagcacccaggccccaaacctgacgcgactctgcgagcgggcacgccgccattttggcttttcggactacaccccccggcccggcgacctcaaacacgagacgacgggggaggcgctgtgtgagcgcctcggcctggacccggaccgcgccctcctgtatctggtcgttaccgagggcttcaaggaggccgtgtgcatcaacaacacctttctgcacctgggaggctcggacaaggtaaccataggcggggcggaggtgcaccgcatacccgtgtatccgttgcagctgttcatgccggattttagccgggtcatcgccgagccgttcaacgccaaccaccgatcgatcggggagaattttacctacccgcttccgttttttaaccgccccctcaaccgcctcctgttcgaggcggtcgtgggacccgccgccgtggcactgcgatgccgaaacgtggacgccgtggcccgcgcggccgcccacctggcgtttgacgaaaaccacgagggcgccgccctccccgccgacattacgttcacggccttcgaagccagccagggtaagaccccgcggggtgggcgcgacggcggcggcaagggcccggcgggcgggttcgaacagcgcctggcctccgtcatggccggagacgccgccctggccctcgagtctatcgtgtcgatggccgtcttcgacgagccgcccaccgacatctccgcgtggccgctgtgcgagggccaggacacggccgcggcccgcgccaacgccgtcggggcgtacctggcgcgcgccgcgggactcgtgggggccatggtatttagcaccaactcggccctccatctcaccgaggtggacgacgccggtccggcggacccaaaggaccacagcaaaccctccttttaccgcttcttcctcgtgcccgggacccacgtggcggccaacccacaggtggaccgcgagggacacgtggtgcccgggttcgagggtcggcccaccgcgcccctcgtcggcggaacccaggaatttgccggcgagcacctggccatgctgtgtgggttttccccggcgctgctggccaagatgctgttttacctggagcgctgcgacggcggcgtgatcgtcgggcgccaggagatggacgtgtttcgatacgtcgcggactccaaccagaccgacgtgccctgcaacctgtgcaccttcgacacgcgccacgcctgcgtacacacgacgctcatgcgcctccgggcgcgccatcccaagttcgccagcgccgcccgcggagccatcggcgtcttcgggaccatgaacagcatgtacagcgactgcgacgtgctgggaaactacgccgccttctcggccctgaagcgcgcggacggatccgagaccgcccggaccatcatgcaggagacgtaccgcgcggcgaccgagcgcgtcatggccgaactcgagaccctgcagtacgtggaccaggcggtccccacggccatggggcggctggagaccatcatcaccaaccgcgaggccctgcatacggtggtgaacaacgtcaggcaggtcgtggaccgcgaggtggagcagctgatgcgcaacctggtggaggggaggaacttcaagtttcgcgacggtctgggcgaggccaaccacgccatgtccctgacgctggacccgtacgcgtgcgggccatgccccctgcttcagcttctcgggcggcgatccaacctcgccgtgtatcaggacctggccctgagccagtgccacggggtgttcgccgggcagtcggtcgaggggcgcaactttcgcaatcaattccaaccggtgctgcggcggcgcgtgatggacatgtttaacaacgggtttctgtcggccaaaacgctgacggtcgcgctctcggagggggcggctatctgcgcccccagcctaacggccggccagacggcccccgccgagagcagcttcgagggcgacgttgcccgcgtgaccctggggtttcccaaggagctgcgcgtcaagagccgcgtgttgttcgcgggcgcgagcgccaacgcgtccgaggccgccaaggcgcgggtcgccagcctccagagcgcctaccagaagcccgacaagcgcgtggacatcctcctcggaccgctgggctttctgctgaagcagttccacgcggccatcttccccaacggcaagcccccggggtccaaccagccgaacccgcagtggttctggacggccctccaacgcaaccagcttcccgcccggctcctgtcgcgcgaggacatcgagaccatcgcgttcattaaaaagttttccctggactacggcgcgataaactttattaacctggcccccaacaacgtgagcgagctggcgatgtactacatggcaaaccagattctgcggtactgcgatcactcgacatacttcatcaacaccctcacggccatcatcgcggggtcccgccgtccccccagcgtgcaggcggcggccgcgtggtccgcgcagggcggggcgggcctggaggccggggcccgcgcgctgatggacgccgtggacgcgcatccgggcgcgtggacgtccatgttcgccagctgcaacctgctgcggcccgtcatggcggcgcgccccatggtcgtgttggggttgagcatcagcaaatactacggcatggccggcaacgaccgtgtgtttcaggccgggaactgggccagcctgatgggcggcaaaaacgcgtgcccgctccttatttttgaccgcacccgcaagttcgtcctggcctgtccccgggccgggtttgtgtgcgcggcctcgaacctcggcggcggagcgcacgaaagctcgctgtgcgagcagctccggggcattatctccgagggcggggcggccgtcgccagtagcgtgttcgtggcgaccgtgaaaagcctggggccccgcacccagcagctgcagatcgaggactggctggcgctcctggaggacgagtacctaagcgaggagatgatggagctgaccgcgcgtgccctggagcgcggcaacggcgagtggtcgacggacgcggccctggaggtggcgcacgaggccgaggccctagtcagccaactcggcaacgccggggaggtgtttaactttggggattttggctgcgaggacgacaacgcgacgccgttcggcggcccgggggccccgggaccggcatttgccggccgcaaacgggcgttccacggggatgacccgtttggggaggggccccccgacaaaaagggagacctgacgttggatatgctgtga

SEQ ID NO: 3 - DNA, 25 bp, Artificial Sequence
  Description of Artificial Sequence Synthetic oligonucleotide
ttttttttttttttttttttttttt

SEQ ID NO: 4 - protein, 50 aa, Herpes simplex virus
LMRLRARHPKFASAARGAIGVFGTMNSMYSDCDVLGNYAAFSALKRADGS

SEQ ID NO: 5 - protein, 50 aa, Herpes simplex virus
LMRLRARHPKFASAARGAIGVFGTMNSAYSDCDVLGNYAAFSALKRADGS

SEQ ID NO: 6 - protein, 51 aa, Varicella-zoster virus
VHRLRQRMPRFGQATRQPIGVFGTMNSQYSDCDPLGNYAPYLILRKPGDQT

SEQ ID NO: 7 - protein, 49 aa, Marek′s disease virus
IKRLVYRLPKFGYQMRGAMGLFGAMTNNYCDVNALGSYAQFSTLKRSEG

SEQ ID NO: 8 - protein, 48 aa, Epstein-Barr virus
FFRLRDRFPPVMSTQRRDPYVISGASGSYNETDFLGNFLNFIDKEDDG

SEQ ID NO: 9 - protein, 59 aa, Human cytomegalovirus
FVRVRTRLPVVPKQPKKEPCVITVQSRFLNDVDILGSFGRRYNVDAKDGGLDGKGDDGV

SEQ ID NO: 10 - protein, 53 aa, Murine cytomegalovirus
FIRVNTRLPGIPKQIKKEPVVVTLLSRAFADADLLGNYGKRYGLESREAGDGG

SEQ ID NO: 11 - protein, 50 aa, Human herpesvirus
LIRINSRLPQISKTTKKEPIVMTMFSRFYADVDVLGSFGKKGVNESKDPM

SEQ ID NO: 12 - protein, 48 aa, Kaposi′s sarcoma-associated herpesvirus
FYRMKDRFPPVLSNVKRDPYVITGTAGTYNDLEILGNFATFREREEEG

SEQ ID NO: 13 - protein, 57 aa, Herpes simplex virus
AGFVCAASNLGGGAHESSLCEQLRGIISEGGAAVASSVFVATVKSLGPRTQQLQIED

SEQ ID NO: 14 - protein, 57 aa, Herpes simplex virus
AGFVCAASSLGGGAHEHSLCEQLRGIIAEGGAAVASSVFVATVKSLGPRTQQLQIED

SEQ ID NO: 15 - protein, 57 aa, Varicella-zoster virus
GGFICPVTGPSSGNRETTLSDQVRGIIVSGGAMVQLAIYATVVRAVGARAQHMAFDD

SEQ ID NO: 16 - protein, 57 aa, Marek′s disease virus
VGFVAEQPIFSSGIKETTLIDRVRMVLSEESAAPHAAVYMLALKMVGDRVRQMELED

SEQ ID NO: 17 - protein, 59 aa, Epstein-Barr virus
LGLTVKRRTQAATTYEIENIRAGLEAIISQKQEEDCVFDVVCNLVDAMGEACASLTRDD

SEQ ID NO: 18 - protein, 57 aa, Human cytomegalovirus
ADVLIKRSRRENVLFDADLIKNRVMLALDAENLDCDPEVMAVYEILSVREEIPASDD

SEQ ID NO: 19 - protein, 59 aa, Murine cytomegalovirus
VDVLIRRSRRENVMYDRDVVRSRVLAALDSTGLDVDPELAAIAELMEGRDEGDIPEIDD

SEQ ID NO: 20 - protein, 57 aa, Human herpesvirus
CDIIVKKVKREAIVYDVEVIRGKVLNIIESLSNSVNPELLILAEVMKDRDSKPTMDD

SEQ ID NO: 21 - protein, 60 aa, Kaposi′s sarcoma-associated herpesvirus
VDRLVKRIVGINSGEFEAEAVKRSVQNVLEDRDNPNLPKTVVLELVKHLGSSCASLTEED

SEQ ID NO: 22 - protein, 1196 aa, Herpes simplex virus
MDTKPKTTTTVKVPPGPMGYVYGRACPAEGLELLSLLSARSGDADVAVAPLIVGLTVESGFEANVAAVVGSRTTGLGGTAVSLKLMPSHYSPSVYVFHGGRHLAPSTQAPNLTRLCERARPHFGFADYAPRPCDLKHETTGDALCERLGLDPDRALLYLVITEGFREAVCISNTFLHLGGMDKVTIGDAEVHRIPVYPLQMFMPDFSRVIADPFNCNHRSIGENFNYPLPFFNRPLARLLFEAVVGPAAVALRARNVDAVARAAAHLAFDENHEGAALPADITFTAFEASQGKPQRGARDAGNKGPAGGFEQRLASVMAGDAALALESIVSMAVFDEPPPDITTWPLLEGQETPAARAGAVGAYLARAAGLVGAMVFSTNSALHLTEVDDAGPADPKDHSKPSFYRFFLVPGTHVAANPQLDREGHVVPGYEGRPTAPLVGGTQEFAGEHLAMLCGFSPALLAKMLFYLERCDGGVIVGRQEMDVFRYVADSGQTDVPCNLCTFETRHACAHTTLMRLRARHPKFASAARGAIGVFGTMNSAYSDCDVLGNYAAFSALKRADGSENTRTIMQETYRAATERVMAELEALQYVDQAVPTALGRLETIIGNREALHTVVNNIKQLVDREVEQLMRNLIEGRNFKFRDGLAEANHAMSLSLDPYTCGPCPLLQLLARRSNLAVYQDLALSQCHGVFAGQSVEGRNFRNQFQPVLRRRVMDLFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAVIFPNGKPPGSNQPNPQWFWTALQRNQLPARLLSREDIETIAFIKRFSLDYGAINFINLAPNNVSELAMYYMANQILRYCDHSTYFINTLTAVIAGSRRPPSVQAAAAWAPQGGAGLEAGARALMDSLDAHPGAWTSMFASCNLLRPVMAARPMVVLGLSISKYYGMAGNDRVFQAGNWASLLGGKNACPLLIFDRTRKFVLACPRAGFVCAASSLGGGAHEHSLCEQLRGIIAEGGAAVASSVFVATVKSLGPRTQQLQIEDWLALLEDEYLSEEMMEFTTRALERGHGEWSTDAALEVAHEAEALVSQLGAAGEVFNFGDFGDEDDHAASFGGLAAAAGAAGVARKRAFHGDDPFGEGPPEKKDLTLDML

SEQ ID NO: 23 - protein, 1204 aa, Varicella-zoster virus
MENTQKTVTVPTGPLGYVYACRVEDLDLEEISFLAARSTDSDLALLPLMRNLTVEKTFTSSLAVVSGARTTGLAGAGITLKLTTSHFYPSVFVFHGGKHVLPSSAAPNLTRACNAARERFGFSRCQGPPVDGAVETTGAEICTRLGLEPENTILYLVVTALFKEAVFMCNVFLHYGGLDIVHINHGDVIRIPLFPVQLFMPDVNRLVPDPFNTHHRSIGEGFVYPTPFYNTGLCHLIHDCVIAPMAVALRVRNVTAVARGAAHLAFDENHEGAVLPPDITYTYFQSSSSGTTTARGARRNDVNSTSKPSPSGGFERRLASIMAADTALHAEVIFNTGIYEETPTDIKEWPMFIGMEGTLPRLNALGSYTARVAGVIGAMVFSPNSALYLTEVEDSGMTEAKDGGPGPSFNRFYQFAGPHLAANPQTDRDGHVLSSQSTGSSNTEFSVDYLALICGFGAPLLARLLFYLERCDAGAFTGGHGDALKYVTGTFDSEIPCSLCEKHTRPVCAHTTVHRLRQRMPRFGQATRQPIGVFGTMNSQYSDCDPLGNYAPYLILRKPGDQTEAAKATMQDTYRATLERLFIDLEQERLLDRGAPCSSEGLSSVIVDHPTFRRILDTLRARIEQTTTQFMKVLVETRDYKIREGLSEATHSMALTFDPYSGAFCPITNFLVKRTHLAVVQDLALSQCHCVFYGQQVEGRNFRNQFQPVLRRRFVDLFNGGFISTRSITVTLSEGPVSAPNPTLGQDAPAGRTFDGDLARVSVEVIRDIRVKNRVVFSGNCTNLSEAARARLVGLASAYQRQEKRVDMLHGALGFLLKQFHGLLFPRGMPPNSKSPNPQWFWTLLQRNQMPADKLTHEEITTIAAVKRFTEEYAAINFINLPPTCIGELAQFYMANLILKYCDHSQYLINTLTSIITGARRPRDPSSVLHWIRKDVTSAADIETQAKALLEKTENLPELWTTAFTSTHLVRAAMNQRPMVVLGISISKYHGAAGNNRVFQAGNWSGLNGGKNVCPLFTFDRTRRFIIACPRGGFICPVTGPSSGNRETTLSDQVRGIIVSGGAMVQLAIYATVVRAVGARAQHMAFDDWLSLTDDEFLARDLEELHDQIIQTLETPWTVEGALEAVKILDEKTTAGDGETPTNLAFNFDSCEPSHDTTSNVLNISGSNISGSTVPGLKRPPEDDELFDLSGIPIKHGNITMEMI

SEQ ID NO: 24 - protein, 1191 aa, Marek′s disease virus
MDGVGKSVKLCGGPIGYIYATPKCSVPVDELAILAAKSNDCDDAVLPLVAGLTVESDFVWNVAAVAGTKTTGLGSGGTTLKLVPTHYHPCVFVFYGGDCIKPCTKAPNLTKACDLARERFGYSAYSSPAPTAFETTGQQICEALEMDAQNVMLYLVVTELFKEVIYLCNSFLHFGGSDVVTINNADVRRIPIYPLHLVLPDFNRITNEPFSEKPRALGEGAIMPKAFYNDSLCRLLHGYVLSTTAVGLRVRNIDAIARGAAHLCFDENHEGTLLPADTTFTAFTPAAETTKGQSKMGKREGSDVSGGGYERRTASLMASDATLAIENVISASVYEDPIPDVNKWPIYCNPVGYADRIEALSAYMARVAGLVGAMVFSSNSVIYMTEVGEAGSTEGKETSTTAPSFYRFFQIAAPHLSANPLVDRDGKPVSGENLSKSTSASQSEYSLDYLILACGFCPQLLARFLFYLERCDGGAKACHHDLDTVKFVSSAIDADMPCELCDKTSRIYCAHTTIKRLVYRLPKFGYQMRGAMGLFGAMTNNYCDVNALGSYAQFSTLKRSEGEASRSVMQDTYRLTVERMMKALEKEGLLTCDDPTNMASADANIRDGNSFIRAISTMKNIIESEASQLMRNLTEIREYNIREGLGDANHTLSLAVEPYASGICPVLAFLSRRTIIAVVQDMALSQCSIVMQGQQVEARNFRTQFQAVLKRRVLELQNAGFITSKTITVTLEDQQICVPDPSKSQYDSVISNMEGDLVKVTVEIFRELKVKNKVLFGGGIAGAASEATKSRLAGMVEAYQRPTKTMHVLNGPLGFAVKRYHTLLFPDVKMPNGATPNALWFWILLLRNQLPAGILSKEEEDKSLFIKKFTKSYADMNYINISPTCFGDLAQFYLANTILKYCSHKHFFINTISALVAVSRRPRDPAIVLPWIERPITKGQDVAPAAQQLIASMSDHKDIWCATFSSTNLVGSIMTTKPFVVIGISISKYHGMAGSTKVFQSGNWGNIMGGRNVCSLMSFDRTHRYVMTCPRVGFVAEQPIFSSGIKETTLIDRVRMVLSEESAAPHAAVYMLALKMVGDRVRQMELEDWMEITNDEYISSLIDELNKQVEEAEGGWNADAAMTLAKEMVNMAMSIPTDGPTFDFDACDENLEGHADGQTISETNLKRPNMNVFDLEPIPEKRVPVLSVDML

SEQ ID NO: 25 - protein, 1128 aa, Epstein-Barr virus
MQGAQTSEDNLGSQSQPGPCGYIYFYPLATYPLREVATLGTGYAGHRCLTVPLLCGITVEPGFSINVKALHRRPDPNCGLLRATSYHRDIYVFHNAHMVPPIFEGPGLEALCGETREVFGYDAYSALPRESSKPGDFFPEGLDPSAYLGAVAITEAFKERLYSGNLVAIPSLKQEVAVGQSASVRVPLYDKEVFPEGVPQLRQFYNSDLSRCMHEALYTGLAQALRVRRVGKLVELLEKQSLQDQAKVAKVAPLKEFPASTISHPDSGALMIVDSAACELAVSYAPAMLEASHETPASLNYDSWPLFADCEGPEARVAALHRYNASLAPHVSTQIFATNSVLYVSGVSKSTGQGKESLFNSFYMTHGLGTLQEGTWDPCRRPCFSGWGGPDVTGTNGPGNYAVEHLVYAASFSPNLLARYAYYLQFCQGQKSSLTPVPETGSYVAGAAASPMCSLCEGRAPAVCLNTLFFRLRDRFPPVMSTQRRDPYVISGASGSYNETDFLGNFLNFIDKEDDGQRPDDEPRYTYWQLNQNLLERLSRLGIDAEGKLEKEPHGPRDFVKMFKDVDAAVDAEVVQFMNSMAKNNITYKDLVKSCYHVMQYSCNPFAQPACPIFTQLFYRSLLTILQDISLPICMCYENDNPGLGQSPPEWLKGHYQTLCTNFRSLAIDKGVLTAKEAKVVHGEPTCDLPDLDAALQGRVYGRRLPVRMSKVLMLCPRNIKIKNRVVFTGENAALQNSFIKSTTRRENYIINGPYMKFLNTYHKTLFPDTKLSSLYLWHNFSRRRSVPVPSGASAEEYSDLALFVDGGSRAHEESNVIDVVPGNLVTYAKQRLNNAILKACGQTQFYISLIQGLVPRTQSVPARDYPHVLGTRAVESAAAYAEATSSLTATTVVCAATDCLSQVCKARPVVTLPVTINKYTGVNGNNQIFQAGNLGYFMGRGVDRNLLQAPGAGLRKQAGGSSMRKKFVFATPTLGLTVKRRTQAATTYEIENIRAGLEAIISQKQEEDCVFDVVCNLVDAMGEACASLTRDDAEYLLGRFSVLADSVLETLATIASSGIEWTAEAARDFLEGVWGGPGAAQDNFISVAEPVSTASQASAGLLLGGGGQGSGGRRKRRLATVLPGLEV

SEQ ID NO: 26 - protein, 1235 aa, Human cytomegalovirus
MSHEELTALAPVGPAAFLYFSRLNAETQEILATLSLCDRSSSVVIAPLLAGLTVEADFGVSVRTPVLCYDGGVLTKVTSFCPFALYFHHTQGIVAFTEDHGDVHRLCEDARQKYALEAYMPEADRVPTDLAALCAAVGCQASETTVHVVVGNGLKEFLFAGQLIPCVEEATTVRLHGGEAVRVPLYPPTLFNSLQLDAEADEVSLDARSAFVEARGLYVPAVSETLFYYVYTSWCQSLRFSEPRVLIEAALRQFVHDSQQSVKLAPHKRYLGYMSQRLSSLEKDHLMLSDAVVCELAFSFASVFFDSAYQPAESMLFSEWPLVTNATDHRDLIRALTELKLHLSTHVAALVFSANSVLYQHRLVYLQSSARHPSAGGTASQETLLKAIQFTNGLSAACEDVYNDARKVLKFQGAPLKDERYGPQHLALVCGTCPQLVSGFVWYLNRVSVYNTGLSGSSTLTNHLVGCAAGLCEACGGTCCHTCYQTAFVRVRTRLPVVPKQPKKEPCVITVQSRFLNDVDILGSFGRRYNVDAKDGGLDGKGDDGVPGGGAGGGGGRDVSGGPSDGLGGGRGGGGGGDSGGMMGRGGRMLGASVDRTYRLNRILDYCRKMRLIDPVTGEDTFSAHGKSDFVAVFSALNKFVDDEALGFVSEVRLKSSRDEVAGATQAFNLDLNPYAVAFQPLLAYAYFRSVFYVIQNVALITATSYIVDNPLTTNLVSKWMTQHFQSIHGAFSTTSSRKGFLFTKQIKSSKNSDHDRLLDFRLYAQGTYAVVPMEIKLSRLSVPTLIMVRVKNRPIYRAGKGNAGSVFFRRDHVPRRNPAKGCLGFLLYRHHERLFPECGLPCLQFWQKVCSNALPKNVPIGDMGEFNAFVKFLVAVTADYQEHDLLDVAPDCVLSYVESRFHNKFLCYYGFKDYIGSLHGLTTRLTTQNHAQFPHVLGASPRFSSPAEFALHVKGLKTAGVPAPMAATVARESLVRSVFEHRSLVTVPVSVEKYAGINNSKEIYQFGQIGYFSGNGVERSLNVSSMSGQDYRFMRQRYLLATRLADVLIKRSRRENVLFDADLIKNRVMLALDAENLDCDPEVMAVYEILSVREEIPASDDVLFFVDGCEALAASLMDKFAALQEQGVEDFSLENLRRVLDADAQRLTDAAGGEVHDLSALFAPSGVGAASGVGGGGLLLGESVAGNSICFGVPGETGGGCFLVNAGEDEAGGVGGSSGGGGGSGLLPAKRSRL

SEQ ID NO: 27 - protein, 1191 aa, Murine cytomegalovirus
MADDDLSSLAPVAPAVWMFFLKKTRELADIVAAMSLCDKATPVVIAPLLIDLTVDRDFCGAVRTPMSTYEGGVLTKVTSFCPFAFFFHNTDEILDVVEDHGDVVHLCDDARRRFGVQAFSPLANRDRTDVDVLCDELGIAPAEYTGHVVCGNGLKELLYAGQLIPCPEEAVKVQVGAVDGVKVPLYPYTLFSGGADAAHADGPSAAVACDDPWVLEHGFYDPALSEALFYFMFTSWGQSLRVCETSRLIEAGLQQFVEDTQQTVKLTPFKKYHGYTSQKLTAVERDQLMTVDAVCSELAFSYASIYLDSVYEFSTASNFLEWPLVKNAKTHADLLDNLRDFQLHLAKHIAALIFSSNSILYQTRIVFVPSAGKGANSNPSAQDSLLKSIRFFNGLTGMYDDILNDAKKTIRFEGAVGRDEKYSPHHLAYFCGTSPQLFSTLMWFFNRMSIYSTGVTSGDTVFSHIVNAGSKLCGACGGRCCHTCYATSFIRVNTRLPGIPKQIKKEPVVVTLLSRAFADADLLGNYGKRYGLESREAGDGGGGGAGGRTDEVAAGPPAGGASGLNFVSVDRMKYLGQVLDYCKKNSLIDAITGEDIINVRTKRDFVATVTALNQTIDDAVCRFAMDVRRSGHGRDEISGSTQSFNLDLSPYATAFSPVLSFQYYRTMFSIIQNLALINAASYVVDNPLTTAQISKWVTLHFQSICGAFGTTPLKKGFLNVKDTKNLKSVEFERIMDFRSFQETGRYRKISTEIKSCKMSVQSLKSCRIKNRPISKTPQSSVFFKKGALQRKNPIKGCLSFLLFRCHEKLFPRCGLSCLEFWQRVLQNSLPRSVNVGKVEDFDNLVRFLLTVTDDYDESDVVDIQPDCLLSYVENRFHNKFLYMFGFRDYMSTIQGMSTRLTPQNHSQFPCLLKDAPKFASIAEYVLHFKKMKLDGVKAPQVATITREPVLKKLFDGRSLVSVSFAVEKYSSSMGTRDVFQFGQIGYYVGSGVDRSLNTGSMGTQDYRFMRYRYIIATKLVDVLIRRSRRENVMYDRDVVRSRVLAALDSTGLDVDPELAAIAELMEGRDEGDIPEIDDILFYVDQQEYIARSMYRKMRSLAERGVTDFSLASLREATATNATAAGSAAGGGGSATEGGGGGAAADESGPMYDFSALFSRRDEAEDVNAGLINGDDVRGDDEFELPSKRSRL

SEQ ID NO: 28 - protein, 1131 aa, Human herpesvirus
MADDNETVVSAPICTAAWLYILPKEQKLIEILTTLSLMEKRKSVVISPLLLNLTVENDFFPTVKTPIINYGGTVITKITSFMPVCFFFHGTDVFLKEAEDHGNLDKLCKQTREKFNLQEFVVNGNRKPVDIGKICESVGRNADDVLCHIVVGNGFKELLFAGLLIPCVEEQIQVQVGECLAIKIPLYSATLFESEETLCIDTCTEFIQENGFYAPQISEVLFYLIFTSWGMTLRFNNTLELIKAGLKQFIQDTEQTVKLAPNKTYHGIPGQKLSPIEKDHLMLVDAVITELTFSYTAEYLDSIYENNQIMNFSEWPIIKSAETHEEKIVELKKLRLHLSSHVAALVFAANSILYSNKLAYISNTKQAFNSAITQETLLRSIQFCNSLSSLNEDFYNDARKLIKCNSSPCKEDKFSAFHLAYACATCPQILSHIIWNLNRMSIYNTNCGNSEIYNHIVNCSSNLCEFCEGKCCHSCIGTALIRINSRLPQISKTTKKEPIVMTMFSRFYADVDVLGSFGKKGVNESKDPMKEAQTTPSLDRFKFLGMIHDYCKKNNLIDAITGEDNLNFKSQNDFVNMINDLIQCIEEAVSKCISEMRKTQTSREQIENCLQSFNIDTTPLSLAFSPFFVFTYYKVILIVLQNLALIIGTGYVVDRPCTGNLISKWLMQQYQSLYGAFYNSHFKKGFLNMKTVKIASNVDMEQYIDFNLFKSGKYAKTSIQAKLCRLSMQCLKDFRVKNRPFNKPNKNTQNNPFFKKVKQKKNPLSGCLSFLLFKYHERLFPNLKISCLEFWQRILLNNMPKTIDIGNVEDMRSFIKFTFRVTNSYDEIDLLDIQPECLLSFIEYYFHNKLLSVLGYRDYLTSLHALTSKLVPQNPMLFPVFLKEHPTFSSVQEYVMHVKKLVGNGLKEPMTASLTKEPNFGSIFTGRSIITFGLMIEKFVSVASRDYFHFGQLGWIAGSGVDRNLNPPSSGLQDFRFMRQKFVIATKLCDIIVKKVKREAIVYDVEVIRGKVLNIIESLSNSVNPELLILAEVMKDRDSKPTMDDMLFYVDGREPLAKSVMNKIQHLTDLNVHDFSLSTLLSVFEEQVEDSAAIYDFSELLVEGNEQGFGILKCEETEHENEEPSLKKARL

SEQ ID NO: 29 - protein, 1132 aa, Kaposi′s sarcoma-associated herpesvirus
MALKGPQTLEENIGSAAPTGPCGYLYAYLTHNFPIGEASLLGNGYPEAKVFSLPLLHGLTVESDFPLNVKAVHKKIDATTASVKLTSYHREAIVFHNTHLFQPIFQGKGLEKLCRESRELFGFSTFVEQQHKGTLWSPEACPQLPCANEIFMAVIVTEGFKERLYGGKLVPVPSQTTPVHIGEHQAFKIPLYDEDLFGPSRAQELCRFYNPDISRYLHDSIFTGIAQALRVKDVSTVIQASERQFVHDQYKIPKLVQAKDFPQCASRGTDGSTLMVIDSLVAELGMSYGLSFIEGPQDSCEVLNYDTWPIFENCETPDARLRALEVWHAEQALHIGAQLFAANSVLYLTRVAKLPQKNQRGDANMYNSFYLQHGLGYLSEATVKENGASAFKGVPVSALDGSSYTLQHLAYASSFSPHLLARMCYYLQFLPHHKNTNSQSYNVVDYVGTAAPSQMCDLCQGQCPAVCINTLFYRMKDRFPPVLSNVKRDPYVITGTAGTYNDLEILGNFATFREREEEGNPVEDAPKYTYWQLCQNITEKLASMGISEGGDALRTLIVDIPSFVKVFKGIDSTVEAELLKFINCMIKNNYNFRENIKSVHHILQFACNVYWQAPCPVFLTLYYKSLLTVIQDICLTSCMMYEQDNPAVGIVPSEWLKMHFQTMWTNFKGACFDKGAITGGELKIVHQSMFCDLFDTDAAIGGMFAPARMQVRIARAMLMVPKTIKIKNRIIFSNSTGAESIQAGFMKPASQRDSYIVGGPYMKFLNALHKTLFPSTKTSALYLWHKIGQTTKNPILPGVSGEHLTELCNYVKASSQAFEEINVLDLVPDTLTSYAKIKLNSSILRACGQTQFYATTLSCLSPVTQLVPAEEYPHVLGPVGLSSPDEYRAKVAGRSVTIVQSTLKQAVSTNGRLRPIITVPLVVNKYTGSNGNTNVFHCANLGYFSGRGVDRNLRPESVPFKKNNVSSMLRKRHVIMTPLVDRLVKRIVGINSGEFEAEAVKRSVQNVLEDRDNPNLPKTVVLELVKHLGSSCASLTEEDVIYYLGPYAVLGDEVLSLLSTVGQAGVPWTAEGVASVIQDIIDDCELQFVGPEEPCLIQGQSVVEELFPSPGVPSLTVGKKRKIASLLSDLDL

(19) United States
(12) Patent Application Publication
(10) Pub. No.: US 2014/0243348 A1
(43) Pub. Date: Aug. 28, 2014

(54) COMPOSITIONS AND METHODS FOR TREATING HERPES VIRUSES

(71) Applicant: President and Fellows of Harvard College, Cambridge, MA (US)

(72) Inventors: David M. Knipe, Auburndale, MA (US); Kevin Bryant, Boston, MA (US); David H. Dreyfus, New Haven, CT (US)

(73) Assignee: PRESIDENT AND FELLOWS OF HARVARD COLLEGE, Cambridge, MA (US)

(21) Appl. No.: 14/161,203

(22) Filed: Jan. 22, 2014

Related U.S. Application Data
(63) Continuation of application No. PCT/US2012/047782, filed on Jul. 22, 2012.
(60) Provisional application No. 61/511,016, filed on Jul. 22, 2011.

(51) Int. Cl. A61K 31/513; A61K 31/195; A61K 31/4985; A61K 31/4375; A61K 31/437; A61K 31/47; A61K 31/4035

(52) U.S. Cl. CPC A61K 31/513; A61K 31/47; A61K 31/195; A61K 31/4035; A61K 31/4375; A61K 31/437; A61K 31/4985
USPC 514/250; 514/269; 514/312; 544/319; 546/156; 514/567; 562/451; 514/417; 548/473; 514/416; 548/472; 514/292; 546/86; 514/300; 546/113; 544/346; 546/123; 435/184; 435/235.1

(57) ABSTRACT

Compositions and methods that are useful for the treatment of herpesvirus infection (including herpes simplex virii) are disclosed. Methods for identifying compounds useful for the treatment of herpesvirus infection are also disclosed.

### RELATED APPLICATIONS

This application is a continuation of PCT Patent Application No. PCT/US2012/047782, filed Jul. 22, 2012, which claims the benefit of and priority to U.S. Provisional Patent Application Ser. No. 61/511,016, filed Jul. 22, 2011. The contents of each of the foregoing applications are incorporated herein by reference in their entirety.

### STATEMENT OF RIGHTS TO INVENTIONS MADE UNDER FEDERALLY SPONSORED RESEARCH

This work was supported by the following grants from the National Institutes of Health, Grant No's: AI 063106 and AI 081477. The government has certain rights in the invention.

### BACKGROUND OF THE INVENTION

Herpes simplex virus 1 and 2 are ubiquitous human pathogens affecting nineteen percent of the adult U.S. population. The herpes virus is an enveloped virus that contains a 152 kb dsDNA genome that includes eighty-four open reading frames. The primary site of herpes infection is the epithelium and the virus also undergoes replication there. When viral particles are released from the epithelium, they can infect local sensory neurons. The viral particle is transported from the sensory axon back to the cell body of the sensory neuron where it can establish a latent infection.

Human infection by these herpes viruses typically results in lifelong latent infections that periodically give rise to clinical lesions or asymptomatic viral shedding. Herpes viruses are a major cause of sexually transmitted disease for which no adequate therapies exist. Because transmission of the virus can occur even in the absence of symptoms, public health measures to control the sexual transmission of the virus have been largely ineffective. In addition, chronic infection with the virus lowers immune function and increases the probability that an infected individual will acquire human immunodeficiency virus (HIV).

Herpes infections can also be transmitted from a mother to her infant during childbirth. The resulting neonatal infections have a fifty percent mortality rate and even when the neonate survives the infection, neurological sequelae are common. Better methods of treating and preventing herpes infection are urgently required.

### SUMMARY OF THE INVENTION

The invention generally provides therapeutic and prophylactic compositions that include an ICP8 or ICP8 homolog inhibitor that reduces or eliminates viral replication of a Herpes virus, including but not limited to herpes simplex virus (HSV) (e.g., HSV-1 or HSV-2) and/or or related double stranded DNA virus.

In one aspect, the invention provides a method of inhibiting Herpes virus (e.g., herpes simplex virus) replication in a cell, the method comprising contacting the cell with an agent that inhibits a DDE recombinase, thereby inhibiting herpes virus replication in the cell.

In another aspect, the invention provides a method of inhibiting Herpes virus (e.g., herpes simplex virus) replication in a cell, the method comprising contacting the cell with an agent that reduces the biological activity of a herpes virus polypeptide having functional and/or structural homology to a human immunodeficiency virus (HIV) integrase, thereby inhibiting herpes virus replication in the cell.

In certain embodiments, the polypeptide is ICP8 or an ICP8 homolog (e.g., a homologous viral recombinase of the herpes virus alpha, beta, gamma family or a related double stranded DNA virus). In certain embodiments, the agent is a small compound that inhibits Human Immunodeficiency Virus (HIV) integrase enzymatic activity. In certain embodiments, the agent is selected from the group consisting of Raltegravir, 118-D-24, L-841411, elvitegrevir, MK-2048, XZ100, XZ99, XZ45, XZ15, XZ49, XZ48, and XZ50; or a derivative or analog thereof.

In another aspect, the invention provides a method of inhibiting Herpes virus (e.g., herpes simplex virus) replication in a cell, the method comprising contacting the cell with Raltegravir or 118-D-24, thereby inhibiting herpes virus replication in the cell.

In another aspect, the invention provides a method of Herpes virus (e.g., herpes simplex virus) replication in a cell, the method comprising contacting the cell with an agent that inhibits Infected Cell Protein 8 (ICP8) biological activity or expression in the cell, thereby inhibiting herpes virus replication in the cell.

In certain embodiments, the agent is selected from the group consisting of Raltegravir, 118-D-24, L-841411, elvitegrevir, MK-2048, XZ100, XZ99, XZ45, XZ15, XZ49, XZ48, and XZ50; or a derivative or analog thereof.

In another aspect, the invention provides a method of treating or preventing a Herpes virus (e.g., herpes simplex virus) infection in a subject, the method comprising administering to the subject an effective amount of an agent that inhibits a DDE recombinase, thereby treating or preventing a herpes virus infection in the subject.

In another aspect, the invention provides a method of treating or preventing a Herpes virus (e.g., herpes simplex virus) infection in a subject, the method comprising administering to the subject an effective amount of an agent that reduces the biological activity of a herpes virus polypeptide having functional and/or structural homology to a human immunodeficiency virus (HIV) integrase, thereby treating or preventing a herpes virus infection in a subject.

In certain embodiments, the agent reduces herpes virus replication. In certain embodiments, the effective amount is sufficient to reduce viral replication by at least about 85% or more.

In other embodiments of the invention the herpes virus is an alphaherpesvirus, a betaherpesvirus, or a gammaherpesvirus. In certain embodiments the herpes virus of the invention is capable of infecting a human cell. In other embodiments the herpes virus of the invention is capable of infecting a non-human mammal cell. In yet other embodiments, the herpes virus is Herpes simplex virus 1 (HSV-1), Herpes simplex virus 2 (HSV-2), Epstein Barr virus (EBV), Cytomegalovirus (CMV), Varicella Zoster Virus (VZV), Herpes lymphotropic virus, Human herpes virus 6 (HHV-6), Human herpes virus 7 (HHV-7), Human herpes virus 8 (HHV-8), or Kaposi's sarcoma-associated herpes virus (KSHV).

In another aspect, the invention provides a method of treating or preventing a Herpes virus (e.g., herpes simplex virus) infection in a subject, the method comprising administering to the subject an effective amount of an agent selected from the group consisting of Raltegravir 118-D-24, XZ100, XZ99, XZ45, XZ15, XZ49, XZ48, and XZ50; or a derivative or analog thereof, thereby treating or preventing a herpes virus infection in the subject.

In yet another aspect, the invention provides a method of inhibiting Herpes virus (e.g., herpes simplex virus) replication in a subject, the method comprising administering to the subject an effective amount of a compound capable of inhibiting a viral DDE recombinase, such that replication of herpes virus in the subject is inhibited.

In certain embodiments of the above methods, the method further comprises identifying the subject as having or at risk of developing a herpes virus infection. In certain embodiments, the method further comprises identifying the subject as testing negative for an HIV infection.

In another aspect, the invention provides a method of treating or preventing a Herpes virus (e.g., herpes simplex virus) infection in a subject, the method comprising

diagnosing the subject as having a herpes virus infection; and

administering to the subject an effective amount of an agent selected from the group consisting of Raltegravir, 118-D-24, XZ100, XZ99, XZ45, XZ15, XZ49, XZ48, and XZ50; or a derivative or analog thereof, thereby treating or preventing a herpes virus infection in the subject.

In certain embodiments, the subject is identified as testing negative for an HIV infection.

In certain embodiments, the effective amount is sufficient to reduce viral replication by at least about 85% or more.

In certain embodiments, the subject is identified as having an acyclovir-resistant herpes virus infection.

In yet another aspect, the invention provides a method of inhibiting the re-activation of a latent Herpes virus (e.g., herpes simplex virus) in a subject, the method comprising administering to a subject identified as having a latent herpes virus infection an effective amount of an agent that reduces the biological activity of a herpes virus polypeptide having functional and/or structural homology to a human immunodeficiency virus (HIV) integrase, thereby inhibiting the re-activation of the latent herpes virus in the subject.

In another aspect, the invention provides a method of reducing the propensity of a subject to acquire an HIV infection, the method comprising: diagnosing the subject as having a herpes virus infection; and administering to the subject an effective amount of an agent selected from the group consisting of Raltegravir, 118-D-24 XZ100, XZ99, XZ45, XZ15, XZ49, XZ48, and XZ50; or a derivative or analog thereof; or an agent that reduces the biological activity of a herpes virus polypeptide having functional and/or structural homology to a human immunodeficiency virus (HIV) integrase, thereby treating or preventing a herpes virus infection in the subject.

In certain embodiments of the methods described above, the herpes virus is HSV1 or HSV2.

In another aspect, the invention provides a pharmaceutical composition comprising an effective amount of an agent selected from the group consisting of Raltegravir, 118-D-24, XZ100, XZ99, XZ45, XZ15, XZ49, XZ48, and XZ50; or a derivative or analog thereof; or an agent that reduces the biological activity of a herpes virus polypeptide having functional and/or structural homology to a human immunodeficiency virus (HIV) integrase formulated for topical administration.

In another aspect, the invention provides an isolated herpes virus comprising an alteration in an ICP8 nucleic acid sequence, wherein the alteration decreases viral replication in a cell.

In another aspect, the invention provides a cell infected with the herpes virus comprising an alteration in an ICP8 nucleic acid sequence, wherein the alteration decreases viral replication in a cell.

In still another aspect, the invention provides an immunogenic composition comprising an effective amount of the herpes virus comprising an alteration in an ICP8 nucleic acid sequence, wherein the alteration decreases viral replication in a cell.

In another aspect, the invention provides a method of generating an HSV-specific immune response in a subject, the method comprising administering to the subject an effective amount of the herpes virus comprising an alteration in an ICP8 nucleic acid sequence, wherein the alteration decreases viral replication in a cell in a pharmaceutically acceptable excipient.

In still another aspect, the invention provides a method of identifying a compound that inhibits Herpes virus (e.g., herpes simplex virus) replication in a cell, the method comprising:

contacting a herpes virus infected cell with a test compound, and comparing viral DDE recombinase activity in said cell relative to a reference, wherein a reduction in DDE recombinase activity in said cell identifies the compound as capable of inhibiting herpes virus replication in a cell.

In yet another aspect, the invention provides a method of identifying a compound that treats or prevents a Herpes virus (e.g., herpes simplex virus) infection in a subject, the method comprising:

contacting a Herpes virus (e.g., herpes simplex virus) infected cell with a compound that inhibits HIV integrase;

and comparing Herpes virus (e.g., herpes simplex virus) replication in said cell with a reference, wherein a compound that inhibits herpes virus replication is identified as useful for treating or preventing herpes virus infection.

In still another aspect, the invention provides a method of identifying a compound that inhibits herpes virus replication in a cell, the method comprising:

a) obtaining a crystal structure of HSV ICP8 or obtaining information relating to the crystal structure of HSV ICP8, and

b) modeling a test compound into or on the crystal structure coordinates to determine whether the compound inhibits HSV ICP8 and inhibits replication of Herpes virus (e.g., herpes simplex virus, HSV) in a cell.

In yet another aspect, the invention features a method of inhibiting recombination mediated by ICP8 or an ICP8 homolog involving contacting the ICP8 or ICP8 homolog with an effective amount of Raltegravir, 118-D-24, L-841411, elvitegrevir, MK-2048, XZ100, XZ99, XZ45, XZ15, XZ49, XZ48, or XZ50; or a derivative or analog thereof.

In another aspect, the invention features a method of inhibiting expression of a herpes virus late gene (or inhibiting production of a herpes virus late gene protein product) involving contacting a cell infected with a herpes virus with an agent that inhibits DDE recombinase, thereby inhibiting expression of the herpes virus late gene.

In one embodiment the agent is Raltegravir, 118-D-24, L-841411, elvitegrevir, MK-2048, XZ100, XZ99, XZ45, XZ15, XZ49, XZ48, or XZ50; or a derivative or analog thereof.

By “ICP8 polypeptide” is meant a protein having at least about 85% identity to NCBI Accession No. P17470, or a fragment thereof, having recombinase activity and/or DNA binding activity. In certain embodiments, an ICP8 polypeptide has amino acid sequence identity to NCBI Accession No. BAA01507.1. In other embodiments, a fragment of ICP8 comprises an ICP8 DDE domain or DNA binding domain.

HSV-1 wild type strain KOS ICP8 amino acid sequence:

METKPKTATTIKVPPGPLGYVYARACPSEGIELLALLSARSGDADVAVAP
LVVGLTVESGFEANVAVVVGSRTTGLGGTAVSLKLTPSHYSSSVYVFHGG
RHLDPSTQAPNLTRLCERARRHFGFSDYTPRPGDLKHETTGEALCERLGL
DPDRALLYLVVTEGFKEAVCINNTFLHLGGSDKVTIGGAEVHRIPVYPLQ
LFMPDFSRVIAEPFNANHRSIGENFTYPLPFFNRPLNRLLFEAVVGPAAV
ALRCRNVDAVARAAAHLAFDENHEGAALPADITFTAFEASQGKTPRGGRD
GGGKGPAGGPEQRLASVMAGDAALALESIVSMAVFDEPPTDISAWPLCEG
QDTAAARANAVGAYLARAAGLVGAMVFSTNSALHLTEVDDAGPADPKDHS
KPSFYRFFLVPGTHVAANPQVDREGHVVPGFEGRPTAPLVGGTQEFAGEH
LAMLCGFSPALLAKMLFYLERCDGGVIVGRQEMDVFRYVADSNQTDVPCN
LCTFDTRHACVHTTLMRLRARHPKFASAARGAIGVFGTMNSMYSDCDVLG
NYAAFSALKRADGSETARTIMQETYRAATERVMAELETLQYVDQAVPTAM
GRLETIITNREALHTVVNNVRQVVDREVEQLMRNLVEGRNFKFRDGLGEA
NHAMSLTLDPYACGPCPLLQLLGRRSNLAVYQDLALSQCHGVFAGQSVEG
RNFRNQFQPVLRRRVMDMFNNGFLSAKTLTVALSEGAAICAPSLTAGQTA
PAESSFEGDVARVTLGFPKELRVKSRVLFAGASANASEAAKARVASLQSA
YQKPDKRVDILLGPLGFLLKQFHAAIFPNGKPPGSNQPNPQWFWTALQRN
QLPARLLSREDIETIAFIKKFSLDYGAINFINLAPNNVSELAMYYMANQI
LRYCDHSTYFINTLTAIIAGSRRPPSVQAAAAWSAQGGAGLEAGARALMD
AVDAHPGAWTSMFASCNLLRPVMAARPMVVLGLSISKYYGMAGNDRVFQA
GNWASLMGGKNACPLLIFDRTRKFVLACPRAGFVCAASNLGGGAHESSLC
EQLRGIISEGGAAVASSVFVATVKSLGPRTQQLQIEDWLALLEDEYLSEE
MMELTARALERGNGEWSTDAALEVAHEAEALVSQLGNAGEVFNFGDFGCE
DDNATPFGGPGAPGPAFAGRKRAFHGDDPFGEGPPDKKGDLTLDML

By “ICP8 DDE domain” is meant a portion of the ICP8 polypeptide having recombinase activity and comprising at least amino acid 1087. It is further contemplated that amino acids 860 and 861 may contribute to the structure and/or activity of the DDE domain. ICP8 DDE domain described herein was based on an analysis of amino acid homology of different recombinase proteins.

By “ICP8 biological activity” is meant DNA binding activity, recombinase activity, or any other activity required for viral replication.

By “ICP8 nucleic acid molecule” is meant any nucleic acid molecule that encodes an ICP8 polypeptide. An exemplary ICP8 nucleotide sequence follows: Nucleotide sequence of the ICP8 open reading frame from HSV-1 wild type strain KOS

atggagacaaagcccaagacggcaaccaccatcaaggtcccccccgggcc
cctgggatacgtgtacgctcgcgcgtgtccgtccgaaggcatcgagcttc
tggcgttactgtcggcgcgcagcggcgatgccgacgtcgccgtggcgccc
ctggtcgtgggcctgaccgtggagagcggctttgaggccaacgtagccgt
ggtcgtgggactcgcacgacggggctcgggggtaccgcggtgtccctgaa
actgacgccatcgcactacagctcgtccgtgtacgtattcacggcggccg
gcacctggaccccagcacccaggccccaaacctgacgcgactctgcgagc
gggcacgccgccattttggcttttcggactacaccccccggcccggcgac
ctcaaacacgagacgacgggggaggcgctgtgtgagcgcctcggcctgga
cccggaccgcgccctcctgtatctggtcgttaccgagggcttcaaggagg
ccgtgtgcatcaacaacacctactgcacctgggaggctcggacaaggtaa
ccataggcggggcggaggtgcaccgcatacccgtgtatccgttgcagctg
acatgccggatatagccgggtcatcgccgagccgttcaacgccaaccacc
gatcgatcggggagaatatacctacccgcaccgttattaaccgccccctc
aaccgcctcctg
ttcgaggcggtcgtgggacccgccgccgtggcactgcgatgccgaaacgt
ggacgccgtggcccgcgcggccgcccacctggcgtagacgaaaaccacga
gggcgccgccctccccgccgacattacgttcacggccacgaagccagcca
gggtaagaccccgcggggtgggcgcgacggcggcggcaagggcccggcgg
gcgggttcgaacagcgcctggcctccgtcatggccggagacgccgccctg
gccctcgagtctatcgtgtcgatggccgtcttcgacgagccgcccaccga
catctccgcgtggccgctgtgcgagggccaggacacggccgcggcccgcg
ccaacgccgtcggggcgtacctggcgcgcgccgcgggactcgtgggggcc
atggtatttagcaccaactcggccctccatctcaccgaggtggacgacgc
cggtccggcggacccaaaggaccacagcaaaccctccttttaccgcttct
tcctcgtgcccgggacccacgtggcggccaacccacaggtggaccgcgag
ggacacgtggtgcccgggttcgagggtcggcccaccgcgcccctcgtcgg
cggaacccaggaatttgccggcgagcacctggccatgctgtgtgggtttt
ccccggcgctgctggccaagatgctgttttacctggagcgctgcgacggc
ggcgtgatcgtcgggcgccaggagatggacgtgtttcgatacgtcgcgga
ctccaaccagaccgacgtgccctgcaacctgtgcaccttcgacacgcgcc
acgcctgcgtacacacgacgctcatgcgcctccgggcgcgccatcccaag
ttcgccagcgccgcccgcggagccatcggcgtcttcgggaccatgaacag
catgtacagcgactgcgacgtgctgggaaactacgccgccttctcggccc
tgaagcgcgcggacggatccgagaccgcccggaccatcatgcaggagacg
taccgcgcggcgaccgagcgcgtcatggccgaactcgagaccctgcagta
cgtggaccaggcggtccccacggccatggggcggctggagaccatcatca
ccaaccgcgaggccctgcatacggtggtgaacaacgtcaggcaggtcgtg
gaccgcgaggtggagcagctgatgcgcaacctggtggaggggaggaactt
caagtttcgcgacggtctgggcgaggccaaccacgccatgtccctgacgc
tggacccgtacgcgtgcgggccatgccccctgcttcagcttctcgggcgg
cgatccaacctcgccgtgtatcaggacctggccctgagccagtgccacgg
ggtgttcgccgggcagtcggtcgaggggcgcaactttcgcaatcaattcc
aaccggtgctgcggcggcgcgtgatggacatgtttaacaacgggtttctg
tcggccaaaacgctgacggtcgcgctctcggagggggcggctatctgcgc
ccccagcctaacggccggccagacggcccccgccgagagcagcttcgagg
gcgacgttgcccgcgtgaccctggggtttcccaaggagctgcgcgtcaag
agccgcgtgttgttcgcgggcgcgagcgccaacgcgtccgaggccgccaa
ggcgcgggtcgccagcctccagagcgcctaccagaagcccgacaagcgcg
tggacatcctcctcggaccgctgggctttctgctgaagcagttccacgcg
gccatcttccccaacggcaagcccccggggtccaaccagccgaacccgca
gtggttctggacggccctccaacgcaaccagcttcccgcccggctcctgt
cgcgcgaggacatcgagaccatcgcgttcattaaaaagttttccctggac
tacggcgcgataaactttattaacctggcccccaacaacgtgagcgagct
ggcgatgtactacatggcaaaccagattctgcggtactgcgatcactcga
catacttcatcaacaccctcacggccatcatcgcggggtcccgccgtccc
cccagcgtgcaggcggcggccgcgtggtccgcgcagggcggggcgggcct
ggaggccggggcccgcgcgctgatggacgccgtggacgcgcatccgggcg
cgtggacgtccatgttcgccagctgcaacctgctgcggcccgtcatggcg
gcgcgccccatggtcgtgttggggttgagcatcagcaaatactacggcat
ggccggcaacgaccgtgtgtttcaggccgggaactgggccagcctgatgg
gcggcaaaaacgcgtgcccgctccttatttttgaccgcacccgcaagttc
gtcctggcctgtccccgggccgggtttgtgtgcgcggcctcgaacctcgg
cggcggagcgcacgaaagctcgctgtgcgagcagctccggggcattatct
ccgagggcggggcggccgtcgccagtagcgtgttcgtggcgaccgtgaaa
agcctggggccccgcacccagcagctgcagatcgaggactggctggcgct
cctggaggacgagtacctaagcgaggagatgatggagctgaccgcgcgtg
ccctggagcgcggcaacggcgagtggtcgacggacgcggccctggaggtg
gcgcacgaggccgaggccctagtcagccaactcggcaacgccggggaggt
gtttaactttggggattttggctgcgaggacgacaacgcgacgccgttcg
gcggcccgggggccccgggaccggcatttgccggccgcaaacgggcgttc
cacggggatgacccgtttggggaggggccccccgacaaaaagggagacct
gacgttggatatgctgtga

By “ICP8 homolog” is meant a viral recombinase of the alphaherpesvirus, betaherpesvirus, gammaherpesvirus, or a related double stranded DNA virus that is homologous to ICP8 from HSV-1. The amino acid sequences of several non-limiting illustrative examples of ICP8 homologs are shown in FIG. 1B.

A “DDE recombinase” is a polypeptide that contains a DDE domain or site, a magnesium ion binding site composed of aspartic acid and glutamic acid amino acids.

A “Herpes virus” is a virus belonging to the Herpesviridae family of DNA viruses, and includes members of the three Herpesviridae subfamilies: Alphaherpesvirinae, Betaherpesvirinae, and Gammaherpesvirinae. A herpes virus can be a human virus affecting humans or a virus affecting non-human animals (e.g., mammals). Illustrative non-limiting examples of herpes virus include Herpes simplex virus Type 1 (HSV-1), Herpes simplex virus Type 2 (HSV-2), Epstein Barr virus (EBV), Cytomegalovirus (CMV), Varicella Zoster Virus (VZV), Herpes lymphotropic virus, Human herpes virus 6 (HHV-6), Human herpes virus 7 (HHV-7), Human herpes virus 8 (HHV-8), and Kaposi's sarcoma-associated herpes virus (KSHV).

By “functional homology” is meant an activity or function that is shared between two or more nucleotides or polypeptides, and which may or may not be associated with a shared or conserved primary nucleic acids or amino acid sequence.

By “structural homology” is meant a three dimensional structure that is shared between two or more nucleic acids or polypeptides, and which may or may not be associated with a shared or conserved primary amino acid or nucleotide sequence.

By “integrase activity” is meant an enzymatic activity that catalyzes the integration of one segment of DNA into another.

A subject is “diagnosed as having a Herpes infection” by methods known in the art. For example, a fluid sample from a blister of a subject may be tested by PCR to detect viral DNA. As another example, a subject may be tested for the presence of antibodies specific to the herpes virus.

A subject is “diagnosed as having HIV” if they test positive by an HIV ELISA, an HIV Western Blot, or by PCR. A subject is “negative for an HIV infection” if they do not test positive on two or more of these tests.

By “agent” is meant any small molecule chemical compound, antibody, nucleic acid molecule, or polypeptide, or fragments thereof. A “small molecule” or “small compound” is a chemical compound, preferably non-peptidic, having a molecular weight of less than about 1000 atomic mass units, in certain embodiments, less than about 800 a.m.u. or less than about 600 a.m.u. or less than about 500 a.m.u. or less than about 400 a.m.u.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “ameliorate” is meant decrease, suppress, attenuate, diminish, arrest, or stabilize the development or progression of a disease.

By “analog” is meant a molecule that is not identical, but has analogous functional or structural features. For example, an analog or derivative of a compound disclosed herein (e.g., analogs or derivatives of raltegravir) have ICP8-binding and/or ICP8-inhibitory activity analogous to the disclosed compound(s); e.g., an analog or derivative of raltegravir has ICP8-inhibitory activity analogous to raltegravir or have a chemical structure analogous to raltegravir. Such analogs are encompasessed within the scope fo the present invention. As a further example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. A polypeptide analog may include an unnatural amino acid.

By “binding to” a molecule is meant having a physicochemical affinity for that molecule. Binding may be measured by any of the methods of the invention, e.g., using an in vitro translation binding assay.

By “computer modeling” is meant the application of a computational program to determine one or more of the following: the location and binding proximity of a ligand to a binding moiety, the occupied space of a bound ligand, the amount of complementary contact surface between a binding moiety and a ligand, the deformation energy of binding of a given ligand to a binding moiety, and some estimate of hydrogen bonding strength, van der Waals interaction, hydrophobic interaction, and/or electrostatic interaction energies between ligand and binding moiety. Computer modeling can also provide comparisons between the features of a model system and a candidate compound. For example, a computer modeling experiment can compare a pharmacophore model of the invention with a candidate compound to assess the fit of the candidate compound with the model.

By a “computer system” is meant the hardware means, software means and data storage means used to analyze atomic coordinate data. The minimum hardware means of the computer-based systems of the present invention comprises a central processing unit (CPU), input means, output means and data storage means. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

By “computer readable media” is meant any media which can be read and accessed directly by a computer e.g. so that the media is suitable for use in the above-mentioned computer system. The media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. An effective amount of a compound described herein may range from about 1 μg/Kg to about 5000 mg/Kg body weight.

The invention provides a number of targets that are useful for the development of highly specific drugs to treat a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fitting”, is meant determining by automatic, or semi-automatic means, interactions between one or more atoms of an agent molecule and one or more atoms or binding sites of DDE domains of ICP8, and determining the extent to which such interactions are stable. Various computer-based methods for fitting are described further herein.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “root mean square deviation” is meant the square root of the arithmetic mean of the squares of the deviations from the mean.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

As used herein, the terms “treat,” “treated,” “treating,” “treatment,” and the like refer to reducing or ameliorating a disorder and/or symptoms associated therewith (e.g. HSV1 or HSV2). By “ameliorate” is meant decrease, suppress, attenuate, diminish, arrest, or stabilize the development or progression of a disease (e.g. infection by a Herpes virus such as HSV1 or HSV2). It will be appreciated that, although not precluded, treating a disorder or condition does not require that the disorder, condition, or symptoms associated therewith be completely eliminated.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A and 1B show the alignment of ICP8 and several ICP8 homolog sequences. Amino acid sequences for HSV-1 ICP8 and ICP8 homologs from representative viruses in each of the three subfamilies of herpesviruses (alphaherpesvirus, betaherpesvirus, and gammaherpesvirus) were aligned using the T-Coffee alignment algorithm (Di Tommaso, P., et al., Nucleic Acids Res., vol. 39, pages W13-W17 (Web Server Version)). The ICP8 homologs included in the analysis were from HSV-1 strain KOS(NCBI accession number P17470), varicella-zoster virus (AEW89446), Marek's disease virus (Q9E6P0), Epstein-Barr virus (P03227), human cytomegalovirus (P17147), murine cytomegalovirus (MCMV) (P30672), human herpesvirus 7(O56282), and Kaposi's sarcoma-associated herpesvirus (ADQ57880). Sites with similar amino acids in 4 or more ICP8 homologs are in black letters highlighted in light gray; sites with identical amino acids in 4 or more ICP8 homologs are in white letters highlighted in black; and sites with identical amino acids in all 9 ICP8 homologs are in white letters highlighted in dark gray. In FIG. 1A, two regions are shown, identifying conserved amino acids at positions 545 and 547 (based on their position in HSV-1 ICP8) and the complete conservation of an aspartic acid residue at position 1087. FIG. 1B shows the full alignment.

FIG. 2 shows a schematic depicting five steps of HSV DNA replication. In Step 1, the origin binding protein, UL9, binds to specific sites at an origin (either oriL or oriS) and starts to unwind the DNA. In Step 2, the single-stranded DNA binding protein, ICP8, is recruited to the unwound DNA. Step 3, UL9 and ICP8 recruit the five remaining replication proteins to the replication forks. In Step 4, DNA synthesis initially proceeds via a theta replication mechanism, but then switches to a rolling-circle replication mechanism as shown in Step 5.

FIG. 3A-3D shows a Western Blot, two bar graphs, and a gel-shift, respectively. FIG. 2A is a Western Blot showing the expression of the E860A/D861A and E1086A/D1087A mutants. FIG. 2B is a bar graph that shows the ability of the E860A/D861A and E1086A/D1087A mutants to complement an ICP8 mutant virus. FIG. 2C shows a bar graph depicting the effect of ICP8 DDE mutant plasmids on complementation of an ICP8 mutant virus. Cells were either mock transfected, transfected with an empty vector (pCIΔ), or transfected with plasmids expressing wild type ICP8, the ICP8 d105 deletion mutant, or with the codons that encode the indicated amino acids in ICP8 mutated to encode alanine. At 24 hours post transfection, the cells were infected with the ICP8 mutant 8lacZ. Viral yield samples were harvested at 24 hours post infection and viral yield was determined by plaque assay. The reported values are percent complementation, relative to cells transfected with the plasmid encoding wild type ICP8. FIG. 2D shows the effect of ICP8 DDE mutation on DNA binding. Wild type and DDE mutant ICP8 were expressed from recombinant baculoviruses and purified from infected Sf21 cells. The indicated concentration of each protein was incubated with 40 fmol radiolabeled oligo(dT)25 DNA and resolved on a 5% native polyacrylamide gel.

FIG. 4A-4C shows a bar graph and two line graphs, respectively. FIG. 3A is a bar graph that shows the effect of an ICP8 DDE mutant on viral yield. FIG. 3B shows a line graph that depicts the effect of an ICP8 DDE mutant on viral DNA levels, as normalized to a GAPDH control. FIG. 3C shows the effect of ICP8 DDE mutant on viral DNA replication. Cells were infected with either wild type virus HSV-1 (strain KOS), the ICP8 DDEm mutant, or the ICP8 mutant pm1.a, which is defective for DNA binding and therefore defective for replication of viral DNA. Total DNA was harvested at the times indicated, and viral DNA levels in each sample was determined by real time PCR and normalized to the levels of cellular DNA.

FIG. 5 shows a Western Blot (top) and a Northern Blot (bottom). The Western Blot shows the effect of ICP8 DDE mutant on viral protein expression. The Northern Blot shows the effect of ICP8 DDE mutant on viral gene expression. Cells were infected with either wild type HSV-1 or the ICP8 DDEm mutant, and total RNA was purified from cells at the indicated times. RNA was resolved by agarose gel electrophoresis, transferred to a charged nylon membrane, and probes for representative immediate-early (ICP27), early (ICP8), and late (gC) were hybridized to the membrane.

FIG. 6 is a bar graph that depicts the effects of Raltegravir and 118-D-24 on viral replication at 100 μM and 1 mM, respectively.

FIG. 7 is a line graph that depicts the effect of the HIV integrase inhibitor 118-D-24 on viral yield. Cells were infected with wild type HSV-1 at a multiplicity of infection of 0.01 plaque forming units per cell and treated with the indicated concentration of the HIV integrase inhibitor 118-D-24. At 48 hours post infection, viral yield samples were harvested and the yield was determined by plaque assay. Values are presented as the percent yield remaining, relative to the DMSO vehicle control treatment.

FIG. 8 is a ribbon diagram depicting the structure of the ICP8 polypeptide.

FIG. 9 is a bar graph that shows the effect of the HIV integrase inhibitors on viral yield. Cells were infected with wild type HSV-1 at a multiplicity of infection of 0.01 plaque-forming units per cell and treated with the indicated concentration of the HIV integrase inhibitor specified. At 48 hours post infection, viral yield samples were harvested and the yield was determined by plaque assay. Values are presented as the percent yield remaining, relative to the DMSO vehicle control treatment.

FIG. 10 is a line graph that depicts the effect of the HIV integrase inhibitor 118-D-24 on viral DNA replication. Cells were infected with wild type HSV-1 and were treated with either 1 mM 118-D-24 or DMSO for the time indicated. Total DNA was harvested at the times indicated, and viral DNA levels in each sample was determined by real time PCR and normalized to the levels of cellular DNA.

FIG. 11 shows a Western Blot depicting the effect of the HIV integrase inhibitor 118-D-24 on viral gene expression. Cells were infected with wild type HSV-1 and were treated with either 1 mM 118-D-24 or DMSO for the time indicated.

Total protein samples were harvested, resolved by SDS-PAGE, transferred to a PVDF membrane, and probed with antibodies for representative immediate-early (ICP27), early (ICP8), and late (gC) gene products. Samples were also probed for cellular GAPDH as a loading control.

FIG. 12 is a graph showing the effects of 118-D-24 derivates on HSV replication.

FIG. 13 is a graph showing the effect of various 118-D-24 derivatives on HSV replication over a range of drug concentrations.

FIGS. 14A-14C are a set of graphs that show the effects of XZ45 on HSV-1 replication in Hep2 cells (FIG. 14A) and normal human foreskin fibroblasts (FIG. 14B). A graph showing the cytotoxicity of XZ45 on Hep2 cells is shown in FIG. 14C.

FIGS. 15A-15C are a set of graphs showing the effects of XZ45 on HSV-1 and HSV-2 replication in Hep2 cells (FIGS. 15A and 15B) and human foreskin fibroblasts (FIG. 15C).

FIG. 16 is a graph showing the effect of XZ45 on human cytomegalovirus (HCMV) replication.

FIGS. 17A and 17B show the effects of XZ45 on viral DNA synthesis (FIG. 17A) and late gene expression (FIG. 17B).

FIGS. 18A-18C show that XZ45 does not decrease ssDNA binding to ICP8 as measured by mobility shift assay (FIG. 18A), ssDNA bead pull down assay (FIG. 18B), and ICP8 binding to ssDNA-cellulose (FIG. 18C).

FIG. 19 is a graph showing that XZ45 inhibits HSV recombination.

FIG. 20 is a gel showing the results of a D-loop assay that demonstrates that XZ45 inhibits HSV recombination.

### DETAILED DESCRIPTION OF THE INVENTION

The invention features compositions and methods that are useful for the treatment and prevention of herpes viruses, including but not limited to Herpes Simplex virus (e.g., HSV-1 and/or HSV-2).

The invention is based, at least in part, on the discovery that HIV integrase inhibitors (e.g., Raltegravir and 118-D-24) also inhibit Herpes Simplex Virus (HSV) replication.

### Herpes Virus

Herpes viruses are enveloped viruses having a double stranded DNA genome that bud from the inner nuclear membrane which has become modified by the insertion of herpes glycoproteins. There are at least 25 viruses in the family Herpesviridae which is currently divided into three subfamilies: alphaherpesvirus, betaherpesvirus, and gammaherpesvirus. Eight or more herpes virus types are known to infect man frequently: Herpes simplex virus 1 (HSV-1), Herpes simplex virus 2 (HSV-2), Epstein Barr virus (EBV), Cytomegalovirus (CMV), Varicella Zoster Virus (VZV), Herpes lymphotropic virus, Human herpes virus 6 (HHV-6), Human herpes virus 7 (HHV-7), Human herpes virus 8 (HHV-8), and Kaposi's sarcoma-associated herpes virus (KSHV).

Herpes simplex virus 1 (HSV-1) is a double stranded DNA virus that replicates its genome in the nucleus of infected cells. The HSV-1 genome encodes seven gene products that are directly involved in the replication of viral DNA, all of which are essential for HSV-1 DNA replication. These proteins are the DNA polymerase (which consists of the catalytic subunit UL30 and its processivity factor UL42), an origin binding protein (UL9), a single-stranded DNA binding protein (ICP8, also known as UL29), and a helicase/primase complex (which consists of the proteins UL5, UL52, and UL8).

Without wishing to be bound be theory, it is likely that HSV DNA replication involves a DNA recombination-based mechanism. In support of this model, viral DNA has been observed as a branched structure in infected cells, indicating that recombination likely occurred to create these molecules, and that recombination would likely be required to resolve them. Homologous recombination of the HSV-1 DNA is also known to occur at high frequency, for example to result in the isomerization of the viral genome, which produces 4 different isomers generated by recombination within the terminal and internal repeat sequences. It is clear that recombination of the viral genome occurs during viral infection. However, the viral and cellular proteins required for recombination, as well as the role recombination plays in the HSV-1 life cycle, have yet to be delineated.

One viral protein proposed to be involved in recombination is ICP8, which is a single stranded DNA binding protein that is necessary for viral DNA replication and that exhibits recombinase activity in vitro. The crystal structure of ICP8 revealed that it shares similarities with enzymes in the DDE family of recombinases, such as RAG-1 and HIV Integrase. These proteins utilize conserved D and E residues to coordinate magnesium ions that are involved in catalyzing their enzymatic activities. ICP8 contains two regions of conserved D and E residues, amino acids E860/D861 and E1086/D1087, which are structurally similar to the active D and E residues of other known DDE recombinases.

As described in the Examples below, a genetic approach was used to determine whether these residues were necessary complement the replication of an ICP8 mutant virus. Mutation of the E860/D861 amino acids (e.g. E860A/D861A) complemented replication of an ICP8 mutant virus to only ˜37% the level of wild type ICP8, and a E1086A/D1087A mutant did not complement above background levels, indicating that both regions are important for ICP8 function. A mutant virus with the E1086A/D1087A mutation in ICP8 was created, and this mutant virus was defective for viral DNA replication and both late gene transcript and protein accumulation. It was further shown that D1087A, as a single mutation, recapitulated the phenotype of the double mutant. Taken together, these results indicate that the DDE residues in ICP8 are important for its function during infection, and likely operate by mediating the previously observed recombinase activity of this viral protein.

ICP8 has been shown to mediate several activities involved in DNA recombination in vitro, including strand exchange and strand invasion. Furthermore, ICP8 has been shown to interact with the HSV-encoded alkaline nuclease UL12, which is proposed to play a role in the initiation and/or the resolution of the DNA recombination mechanism.

ICP8 is a major component of HSV-1 replication compartments, which are nuclear domains where viral DNA replication and late gene expression occur. ICP8 also interacts with several cellular proteins known to be involved in recombination and recruits these proteins to viral replication compartments, where they may play important roles in mediating recombination of the HSV-1 genome.

As described in the working Examples below, conserved residues in ICP8 that share structural homology with catalytic residues of enzymes in the DDE family of recombinases, including most notably RAG-1, have been identified and shown to be important for ICP8-mediated recombination. Enzymes in the DDE recombinase family perform recombination reactions using a catalytic triad of aspartic acid (D) and glutamic acid (E) residues that coordinate divalent metal cations. As described in the working Examples below, these putative DDE residues in ICP8 are important for its activity, and a mutant virus with DDE residues in ICP8 mutated is defective for viral DNA replication. These results identify ICP8 residues that are likely required for HSV-1 DNA recombination and indicate that recombination of the viral genome is likely required for viral DNA replication.

Several other viruses encode proteins that contain DDE motifs, such as HIV integrase and HCMV UL89. Antiviral compounds have been developed to inhibit the activity of the HIV integrase enzyme, and one of these compounds, Raltegravir, can also inhibit HCMV UL89 activity. Surprisingly, as reported herein, these compounds, including L-841411, Raltegravir, and 118-D-24, inhibit HSV replication, likely by inhibiting the virally encoded HSV DDE recombinase, ICP8.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof due to viral infection (e.g., with HSV1 or HSV2). Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which Raltegravir, 118-D-24, L-841411, elvitegrevir, or MK-2048 may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with herpes, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Compounds of the Invention

Compounds of the invention were found to inhibit Herpes virus replication, and in particular HSV replication. Without wishing to be bound by any particular theory, these compounds may be particularly effective for the treatment of HSV. In one approach, compounds useful for the treatment of HSV are selected using a molecular docking program to identify compounds that are expected to bind to an ICP8 DDE domain. In certain embodiments, a compound of the invention can bind to ICP8 and reduce ICP8 biological activity and/or disrupt HSV replication.

In certain embodiments, a compound of the invention can prevent, inhibit, disrupt, or reduce by at least 10%, 25%, 50%, 75%, or 100% of the expression and/or biological activity of ICP8.

In certain embodiments, a compound of the invention is a small molecule having a molecular weight less than about 1000 daltons, less than 800, less than 600, less than 500, less than 400, or less than about 300 daltons. Examples of compounds of the invention include Raltegravir, 118-D-24, Elvitegravir (also known as GS 9137 or JTK-303), dolutegravir, MK-2048, L841411, XZ100, XZ99, XZ45, XZ15, XZ49, XZ48, and XZ50 and pharmaceutically acceptable salts thereof. Compounds of the invention also include analogs or derivatives of compounds disclosed herein.

The term “pharmaceutically acceptable salt” also refers to a salt prepared from a compound of the invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like. The term “pharmaceutically acceptable salt” also refers to a salt prepared from a compound disclosed herein or any other compound delineated herein, having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

### In Silico Screening Methods and Systems

In another aspect, the invention provides a machine readable storage medium which comprises the structural coordinates of an ICP8 polypeptide (e.g., ICP8 DDE domain or an amino acid corresponding to positions 547, 623, 645, 735, 860, 861, 1086, and 1087 of HSV protein ICP8). A storage medium encoded with these data is capable of displaying a three-dimensional graphical representation of a molecule or molecular complex which comprises such binding sites on a computer screen or similar viewing device.

The invention also provides methods for designing, evaluating and identifying compounds that bind to the aforementioned binding site. Such compounds are expected to inhibit HSV replication. The invention provides a computer for producing a) a three-dimensional representation of a molecule or molecular complex, wherein said molecule or molecular complex comprises a binding site; or b) a three-dimensional representation of a homologue of said molecule or molecular complex, wherein said homologue comprises a binding site that has a root mean square deviation from the backbone atoms of said amino acids of not more than about 2.0 (more preferably not more than 1.5) angstroms, wherein said computer comprises:

(i) a machine-readable data storage medium comprising a data storage material encoded with machine-readable data, wherein said data comprises the structure coordinates of amino acid residues in the ICP8 DDE domain, or other ICP8 binding site;

(ii) a working memory for storing instructions for processing said machine-readable data;

(iii) a central-processing unit coupled to said working memory and to said machine-readable data storage medium for processing said machine readable data into said three-dimensional representation; and

(iv) a display coupled to said central-processing unit for displaying said three-dimensional representation.

Thus, the computer produces a three-dimensional graphical structure of a molecule or a molecular complex which comprises a binding site.

In another embodiment, the invention provides a computer for producing a three-dimensional representation of a molecule or molecular complex defined by structure coordinates of all of the ICP8 amino acids, or a three-dimensional representation of a homologue of said molecule or molecular complex, wherein said homologue comprises a binding site that has a root mean square deviation from the backbone atoms of said amino acids of not more than 2.0 (more preferably not more than 1.5) angstroms.

In exemplary embodiments, the computer or computer system can include components that are conventional in the art, e.g., as disclosed in U.S. Pat. No. 5,978,740 and/or 6,183,121 (incorporated herein by reference). For example, a computer system can includes a computer comprising a central processing unit (“CPU”), a working memory (which may be, e.g., RAM (random-access memory) or “core” memory), a mass storage memory (such as one or more disk drives or CD-ROM drives), one or more cathode-ray tube (CRT) or liquid crystal display (LCD) display terminals, one or more keyboards, one or more input lines, and one or more output lines, all of which are interconnected by a conventional system bus.

Machine-readable data of this invention may be inputted to the computer via the use of a modem or modems connected by a data line. Alternatively or additionally, the input hardware may include CD-ROM drives, disk drives or flash memory. In conjunction with a display terminal, a keyboard may also be used as an input device.

Output hardware coupled to the computer by output lines may similarly be implemented by conventional devices. By way of example, output hardware may include a CRT or LCD display terminal for displaying a graphical representation of a binding pocket of this invention using a program such as QUANTA or PYMOL. Output hardware might also include a printer, or a disk drive to store system output for later use.

In operation, the CPU coordinates the use of the various input and output devices, coordinates data accesses from the mass storage and accesses to and from working memory, and determines the sequence of data processing steps. A number of programs may be used to process the machine-readable data of this invention, including commercially-available software.

A magnetic storage medium for storing machine-readable data according to the invention can be conventional. A magnetic data storage medium can be encoded with a machine-readable data that can be carried out by a system such as the computer system described above. The medium can be a conventional floppy diskette or hard disk, having a suitable substrate which may be conventional, and a suitable coating, which may also be conventional, on one or both sides, containing magnetic domains whose polarity or orientation can be altered magnetically. The medium may also have an opening (not shown) for receiving the spindle of a disk drive or other data storage device.

The magnetic domains of the medium are polarized or oriented so as to encode in a manner which may be conventional, machine readable data such as that described herein, for execution by a system such as the computer system described herein.

An optically-readable data storage medium also can be encoded with machine-readable data, or a set of instructions, which can be carried out by a computer system. The medium can be a conventional compact disk read only memory (CD-ROM) or a rewritable medium such as a magneto-optical disk which is optically readable and magneto-optically writable.

In the case of CD-ROM, as is well known, a disk coating is reflective and is impressed with a plurality of pits to encode the machine-readable data. The arrangement of pits is read by reflecting laser light off the surface of the coating. A protective coating, which preferably is substantially transparent, is provided on top of the reflective coating.

In the case of a magneto-optical disk, as is well known, a data-recording coating has no pits, but has a plurality of magnetic domains whose polarity or orientation can be changed magnetically when heated above a certain temperature, as by a laser. The orientation of the domains can be read by measuring the polarization of laser light reflected from the coating. The arrangement of the domains encodes the data as described above.

Structure data, when used in conjunction with a computer programmed with software to translate those coordinates into the 3-dimensional structure of a molecule or molecular complex comprising a binding pocket may be used for a variety of purposes, such as drug discovery.

For example, the structure encoded by the data may be computationally evaluated for its ability to associate with chemical entities. Chemical entities that associate with a DDE domain or a binding site of an ICP8 protein are expected to inhibit Herpes virus replication (e.g. HSV1 and HSV2), to inhibit ICP8 biological activity, and/or to disrupt ICP8 sub-cellular localization. Such compounds are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention relates to a method for evaluating the potential of a chemical entity to associate with a) a molecule or molecular complex comprising a binding site defined by structure coordinates and/or amino acid positions in ICP8, as described herein, or b) a homologue of said molecule or molecular complex, wherein said homologue comprises a binding pocket that has a root mean square deviation from the backbone atoms of said amino acids of not more than 2.0 (more preferably 1.5) angstroms.

This method comprises the steps of:

i) employing computational means to perform a fitting operation between the chemical entity and a binding site of the ICP8 polypeptide or fragment thereof or molecular complex; and

ii) analyzing the results of the fitting operation to quantify the association between the chemical entity and the binding pocket. This embodiment relates to evaluating the potential of a chemical entity to associate with or bind to a binding site of an ICP8 polypeptide or fragment thereof.

The term “chemical entity”, as used herein, refers to chemical compounds, complexes of at least two chemical compounds, and fragments of such compounds or complexes.

In certain embodiments, the method evaluates the potential of a chemical entity to associate with a molecule or molecular complex defined by structure coordinates of all of the amino acids of an ICP8 protein, as described herein, or a homologue of said molecule or molecular complex having a root mean square deviation from the backbone atoms of said amino acids of not more than 2.0 (more preferably not more than 1.5) angstroms.

In a further embodiment, the structural coordinates one of the binding sites described herein can be utilized in a method for identifying an antagonist of a molecule comprising an ICP8 binding site (e.g., a DDE domain or DNA binding domain). This method comprises the steps of:

a) using the atomic coordinates of ICP8; and

b) employing the three-dimensional structure to design or select the potential agonist or antagonist. One may obtain the compound by any means available. By “obtaining” is meant, for example, synthesizing, buying, or otherwise procuring the agonist or antagonist. If desired, the method further involves contacting the agonist or antagonist with an ICP8 polypeptide or a fragment thereof to determine the ability of the potential agonist or antagonist to interact with the molecule. If desired, the method also further involves the step of contacting a Herpes infected with an ICP8 binding compound and evaluating inhibition of viral replication, evaluating viral DNA production, cell death, ICP8 biological activity, ICP8 DNA binding activity, ICP8 recombinase activity, ICP8 expression and/or levels, or ICP8 subcellular localization.

In another embodiment, the invention provides a method for identifying a potential agonist or antagonist of an ICP8 polypeptide, the method comprising the steps of:

a) using the atomic coordinates of the ICP8 polypeptide (e.g., DDE domain or DNA binding domain); and

b) employing the three-dimensional structure to design or select the potential agonist or antagonist.

The present inventors' elucidation of heretofore unidentified binding sites of ICP8 polypeptides provides the necessary information for designing new chemical entities and compounds that may interact with ICP8 proteins, in whole or in part, and may therefore modulate (e.g., inhibit) the activity of ICP8 proteins.

The design of compounds that bind to an ICP8 DDE domain sequence, that are cytotoxic to a cell infected with Herpes (e.g. HSV1 or HSV2), that reduce ICP8 expression and/or levels or biological activity, or that disrupt ICP8 subcellular localization, according to this invention generally involves consideration of several factors. In one embodiment, the compound physically and/or structurally associates with at least a fragment of an ICP8 polypeptide, such as a binding site within a DDE domain sequence. Non-covalent molecular interactions important in this association include hydrogen bonding, van der Waals interactions, hydrophobic interactions and electrostatic interactions. Desirably, the compound assumes a conformation that allows it to associate with the ICP8 binding site(s) directly. Although certain portions of the compound may not directly participate in these associations, those portions of the entity may still influence the overall conformation of the molecule. This, in turn, may have a significant impact on the compound's potency. Such conformational requirements include the overall three-dimensional structure and orientation of the chemical compound in relation to all or a portion of the binding site, or the spacing between functional groups comprising several chemical compound that directly interact with the binding site or a homologue thereof.

The potential inhibitory or binding effect of a chemical compound on an ICP8 binding site may be analyzed prior to its actual synthesis and testing by the use of computer modeling techniques. If the theoretical structure of the given compound suggests insufficient interaction and association between it and the target binding site, testing of the compound is obviated. However, if computer modeling indicates a strong interaction, the molecule is synthesized and tested for its ability to bind a DDE domain sequence and/or a DNA binding domain sequence, or to test its biological activity by assaying for example, viral replication by a Herpes infected cell (e.g. a cell infected with HSV1 or HSV2), by assaying a reduction in ICP8 expression and/or levels or biological activity, or by assaying ICP8 subcellular localization. Candidate compounds may be computationally evaluated by means of a series of steps in which chemical entities or fragments are screened and selected for their ability to associate with the ICP8 DDE domain and/or DNA binding domain.

One skilled in the art may use one of several methods to screen chemical compounds, or fragments for their ability to associate with an ICP8 binding site. This process may begin by visual inspection of, for example, an ICP8 binding site on the computer screen based on the ICP8 structure coordinates described herein, or other coordinates which define a similar shape generated from the machine-readable storage medium. Selected fragments or chemical compounds are then positioned in a variety of orientations, or docked, within that binding site as defined supra. Docking may be accomplished using software such as Quanta and DOCK, followed by energy minimization and molecular dynamics with standard molecular mechanics force fields, such as CHARMM and AMBER.

Specialized computer programs (e.g., as known in the art and/or commercially available and/or as described herein) may also assist in the process of selecting fragments or chemical entities.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target binding site.

Instead of proceeding to build an inhibitor of a binding pocket in a step-wise fashion one fragment or chemical entity at a time as described above, inhibitory or other binding compounds may be designed as a whole or “de novo” using either an empty binding site or optionally including some portion(s) of a known inhibitor(s). There are many de novo ligand design methods known in the art, some of which are commercially available (e.g., LeapFrog, available from Tripos Associates, St. Louis, Mo.).

Other molecular modeling techniques may also be employed in accordance with this invention (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)).

Once a compound has been designed or selected, the efficiency with which that entity may bind to a binding site may be tested and optimized by computational evaluation.

Specific computer software is available in the art to evaluate compound deformation energy and electrostatic interactions. Examples of programs designed for such uses include: AMBER; QUANTA/CHARMM (Accelrys, Inc., Madison, Wis.) and the like. These programs may be implemented, for instance, using a commercially-available graphics workstation. Other hardware systems and software packages will be known to those skilled in the art.

Another technique involves the in silico screening of virtual libraries of compounds, e.g., as described herein (see, e.g., Examples). Many thousands of compounds can be rapidly screened and the best virtual compounds can be selected for further screening (e.g., by synthesis and in vitro or in vivo testing). Small molecule databases can be screened for chemical entities or compounds that can bind, in whole or in part, to an ICP8 DDE domain and/or DNA binding site. In this screening, the quality of fit of such entities to the binding site may be judged either by shape complementarity or by estimated interaction energy.

A computer for producing a three-dimensional representation of:

a) a molecule or molecular complex, wherein said molecule or molecular complex comprises a DDE domain and/or a DNA binding domain of an ICP8 polypeptide defined by structure coordinates of amino acid residues in DDE domain and/or a DNA binding domain of an ICP8 polypeptide; or

b) a three-dimensional representation of a homologue of said molecule or molecular complex, wherein said homologue comprises a binding site that has a root mean square deviation from the backbone atoms of said amino acids of not more than about 2.0 (more preferably not more than 1.5) angstroms, wherein said computer comprises:

(i) a machine-readable data storage medium comprising a data storage material encoded with machine-readable data, wherein said data comprises the structure coordinates of structure coordinates of amino acid residues in the DDE domain and/or a DNA binding domain of an ICP8 polypeptide;

(ii) a working memory for storing instructions for processing said machine-readable data;

(iii) a central-processing unit coupled to said working memory and to said machine-readable data storage medium for processing said machine readable data into said three-dimensional representation; and

(iv) a display coupled to said central-processing unit for displaying said three-dimensional representation. As described in the Examples, compounds identified using in silico methods may optionally be tested in vitro or in vivo, for example, using the “Additional Screening Methods” described below, or any other method known in the art.

### Additional Screening Methods

As described above, the invention provides specific examples of chemical compounds, including, but not limited to, Raltegravir, L-841411, 118-D-24, Elvitegravir (also known as GS 9137 or JTK-303), dolutegravir, and MK-2048, that inhibit the biological activity (e.g. recombinase activity and/or DNA binding activity) of an ICP8 polypeptide, as well as the replication of a Herpes virus (e.g. HSV1 or HSV2). However, the invention is not so limited. The invention further provides a simple means for identifying agents (including nucleic acids, peptides, small molecule inhibitors, and mimetics) that are capable of binding to an ICP8 polypeptide, that can inhibit viral replication in an infected cell, that reduce ICP8 expression and/or levels or biological activity, or that disrupt ICP8 subcellular localization. Such compounds are also expected to be useful for the treatment or prevention of a Herpes infection.

Virtually any agent that specifically binds to an ICP8 polypeptide or that modulates ICP8 expression and/or levels or biological activity may be employed in the methods of the invention. Methods of the invention are useful for the high-throughput low-cost screening of candidate agents that reduce, slow, or eliminate replication of a Herpes virus in an infected cell, in particular a cell infected with HSV1 and/or HSV2. A candidate agent that specifically binds to ICP8 is then isolated and tested for activity in an in vitro assay or in vivo assay for its ability to reduce Herpes viral replication, reduce ICP8 recombinase activity, or reduce ICP8 DNA binding activity. One skilled in the art appreciates that the effects of a candidate agent on a cell is typically compared to a corresponding control cell not contacted with the candidate agent. Thus, the screening methods include comparing the proliferation of a virus in an infected cell contacted by a candidate agent to the proliferation of an untreated control cell.

In other embodiments, the expression or activity of ICP8 in a cell treated with a candidate agent is compared to untreated control samples to identify a candidate compound that decreases the expression or biological activity of an ICP8 polypeptide in the contacted cell. Polypeptide expression or activity can be compared by procedures well known in the art, such as Western blotting, flow cytometry, immunocytochemistry, binding to magnetic and/or ICP8-specific antibody-coated beads, in situ hybridization, fluorescence in situ hybridization (FISH), ELISA, microarray analysis, RT-PCR, Northern blotting, or colorimetric assays, such as the Bradford Assay and Lowry Assay.

In one working example, one or more candidate agents are added at varying concentrations to the culture medium containing a Herpes infected cell. An agent that reduces the expression of an ICP8 or gC polypeptide expressed in the cell, or viral DNA replication, is considered useful in the invention; such an agent may be used, for example, as a therapeutic to prevent, delay, ameliorate, stabilize, or treat a Herpes infection of a cell. Once identified, agents of the invention (e.g., agents that specifically bind to and/or antagonize ICP8) may be used to treat a Herpes infected cell. An agent identified according to a method of the invention is locally or systemically delivered to treat a Herpes infection in situ.

In one embodiment, the effect of a candidate agent may, in the alternative, be measured at the level of ICP8 polypeptide production using the same general approach and standard immunological techniques, such as Western blotting or immunoprecipitation with an antibody specific for ICP8. For example, immunoassays may be used to detect or monitor the expression of ICP8 in a Herpes infected cell. In one embodiment, the invention identifies a polyclonal or monoclonal antibody (produced as described herein) that is capable of binding to and blocking the biological activity or disrupting the subcellular localization of an ICP8 polypeptide. A compound that disrupts the subcellular localization, or reduces the expression or activity of an ICP8 polypeptide is considered particularly useful. Again, such an agent may be used, for example, as a therapeutic to prevent or treat a Herpes infection.

Alternatively, or in addition, candidate compounds may be identified by first assaying those that specifically bind to and antagonize an ICP8 polypeptide of the invention and subsequently testing their effect on a Herpes infected cells as described in the Examples. In one embodiment, the efficacy of a candidate agent is dependent upon its ability to interact with the ICP8 polypeptide. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate Herpes viral replication may be assayed by any standard assays (e.g., those described herein). In one embodiment, viral replication is determined by a viral replication assay, or a viral DNA replication assay. In another embodiment, ICP8 expression is monitored immunohistochemically.

Potential ICP8 antagonists include organic molecules, peptides, peptide mimetics, polypeptides, nucleic acid ligands, aptamers, and antibodies that bind to an ICP8 polypeptide and reduce its activity. In one particular example, a candidate compound that binds to an ICP8 polypeptide may be identified using a chromatography-based technique. For example, a recombinant ICP8 polypeptide of the invention may be purified by standard techniques from cells engineered to express the polypeptide, or may be chemically synthesized, once purified the peptide is immobilized on a column. A solution of candidate agents is then passed through the column, and an agent that specifically binds the ICP8 polypeptide or a fragment thereof is identified on the basis of its ability to bind to an ICP8 polypeptide and to be immobilized on the column. To isolate the agent, the column is washed to remove non-specifically bound molecules, and the agent of interest is then released from the column and collected. Agents isolated by this method (or any other appropriate method) may, if desired, be further purified (e.g., by high performance liquid chromatography). In addition, these candidate agents may be tested for their ability to reduce Herpes replication. Agents isolated by this approach may also be used, for example, as therapeutics to treat or prevent a Herpes infection. Compounds that are identified as binding to an ICP8 polypeptide with an affinity constant less than or equal to 1 nM, 5 nM, 10 nM, 100 nM, 1 μM or 10 μM are considered particularly useful in the invention.

### Test Compounds and Extracts

In general, ICP8 antagonists (e.g., agents that specifically bind and reduce the activity of an ICP8 polypeptide) are identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known those known as therapeutics for the treatment of other types of viral infection (e.g. HIV). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have an ICP8 binding activity, further fractionation of the positive lead extract is necessary to isolate molecular constituents responsible for the observed effect. Thus, the goal of the extraction, fractionation, and purification process is the careful characterization and identification of a chemical entity within the crude extract that reduces ICP8 recombinase activity, DNA binding activity, and/or Herpes viral replication. Methods of fractionation and purification of such heterogeneous extracts are known in the art. If desired, compounds shown to be useful as therapeutics are chemically modified according to methods known in the art.

The present invention provides methods of treating disease (e.g. Herpes infection) and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a disease (e.g. Herpes infection) or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which a Herpes infection may be implicated.

In one embodiment, the invention provides a method of monitoring treatment progress. The method includes the step of determining a level of diagnostic marker (Marker) (e.g. a Herpes polypeptide such as ICP8 or gC, or any target delineated herein modulated by a compound herein, a protein or indicator thereof, etc.) or diagnostic measurement (e.g., screen, assay) in a subject suffering from or susceptible to a disorder or symptoms thereof associated with a Herpes infection, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Pharmaceutical Therapeutics

In other embodiments, agents discovered to have medicinal value using the methods described herein are useful as a drug or as information for structural modification of existing compounds, e.g., by rational drug design. Such methods are useful for screening agents having an effect on an ICP8 recombinase or DNA binding activity, or Herpes viral replication.

For therapeutic uses, the compositions or agents identified using the methods disclosed herein may be administered systemically, for example, formulated in a pharmaceutically-acceptable buffer such as physiological saline. Preferable routes of administration include, for example, oral, subcutaneous, intravenous, interperitoneally, intramuscular, or intradermal injections that provide continuous, sustained levels of the drug in the patient. In certain embodiments, the route of administration is oral administration; in other embodiments, topical administration is preferred. Compounds of the invention can be administered by a combination of routes, such as combined oral and topical administration.

Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the Herpes infection. Generally, amounts will be in the range of those used for other agents used in the treatment of other diseases associated with Herpes infections, or infection by other similar viruses, although in certain instances lower amounts will be needed because of the increased specificity of the compound. A compound is administered at a dosage that inhibits Herpes viral replication, or that reduces ICP8 expression and/or levels or biological activity as determined by a method known to one skilled in the art, or using any assay that measures the expression or the biological activity of an ICP8 polypeptide.

### Formulation of Pharmaceutical Compositions

The administration of a compound for the treatment of a Herpes infection may be by any suitable means that results in a concentration of the therapeutic that, combined with other components, is effective in ameliorating, reducing, or stabilizing a Herpes infection. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight, per day. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight, per day. In other embodiments, it is envisaged that doses may be in the range of about 5 mg compound/Kg body to about 20 mg compound/Kg body. In other embodiments the doses may be about 8, 10, 12, 14, 16 or 18 mg/Kg body weight, per day. In certain embodiments, the dose may be selected to provide a concentration is a body fluid of the subject (e.g., blood, lymph, saliva, etc.) from about 10 μM to about 10 mM, or from about 100 μM to about 1 mM. Doses may be administered once per day, or in divided doses, e.g., twice per day, three times per day, or more frequently as needed. Of course, this dosage amount may be adjusted upward or downward, as is routinely done in such treatment protocols, depending on the results of the initial clinical trials and the needs of a particular patient.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a Herpes infection by using carriers or chemical derivatives to deliver the therapeutic agent to a particular cell type (e.g. sensory neurons). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a Herpes infection, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutaminine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active anti-Herpes therapeutic substance). The coating may be applied on the solid dosage form in a similar manner as that described in Encyclopedia of Pharmaceutical Technology, supra.

At least two anti-Herpes therapeutics may be mixed together in the tablet, or may be partitioned. In one example, the first active anti-Herpes therapeutic is contained on the inside of the tablet, and the second active anti-Herpes therapeutic is on the outside, such that a substantial portion of the second anti-Herpes therapeutic is released prior to the release of the first anti-Herpes therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active anti-Herpes therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Combination Therapies

Optionally, an anti-Herpes therapeutic may be administered in combination with any other standard anti-Herpes therapy; such methods are known to the skilled artisan and described in Remington's Pharmaceutical Sciences by E. W. Martin. If desired, agents of the invention (including Raltegravir, 118-D-24, Elvitegravir (also known as GS 9137 or JTK-303), dolutegravir, MK-2048, L841411, and pharmaceutically acceptable salts thereof) are administered in combination with any conventional anti-neoplastic therapy, including but not limited to, surgery, radiation therapy, or chemotherapy. In one preferred embodiment, an agent of the invention is administered in combination with temozolomide.

### Kits or Pharmaceutical Systems

The present compositions may be assembled into kits or pharmaceutical systems for use in ameliorating a Herpes infection. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### ICP8 with Variations of the DDE Residues have Decreased Ability to Complement Replication of an ICP8 Mutant Virus

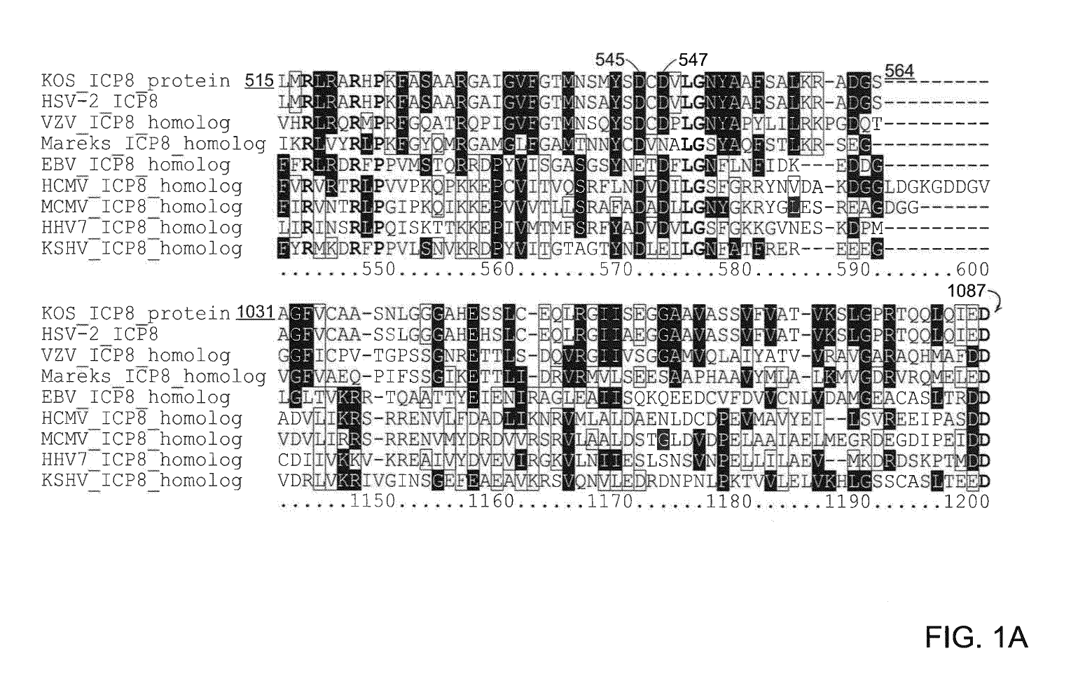

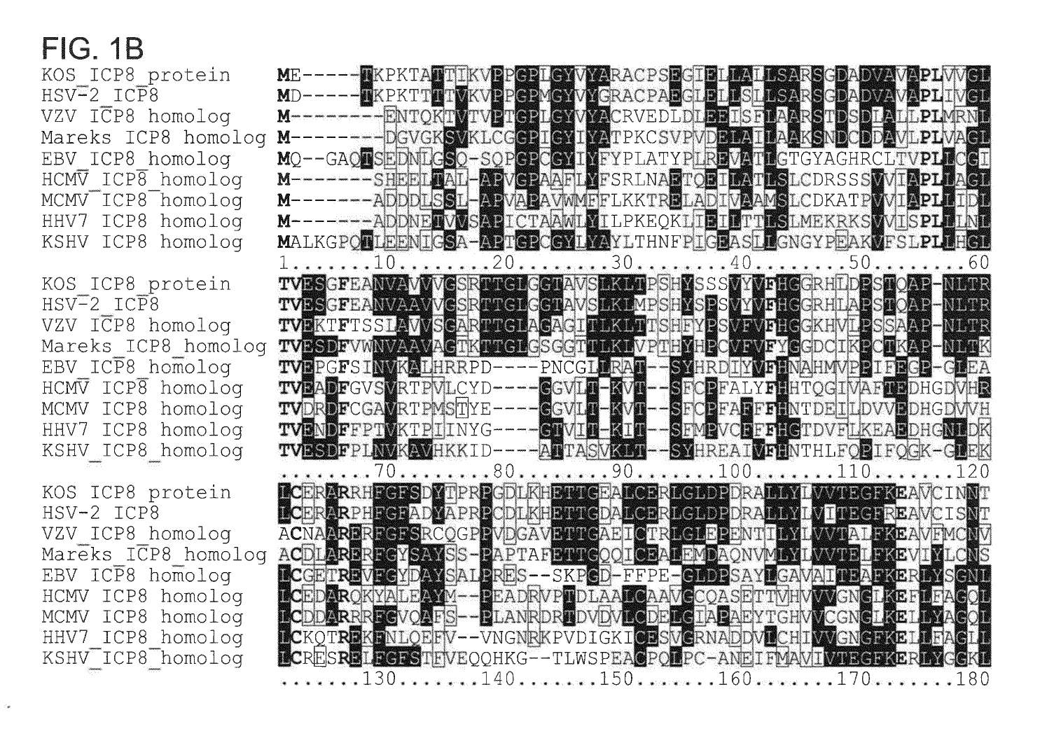

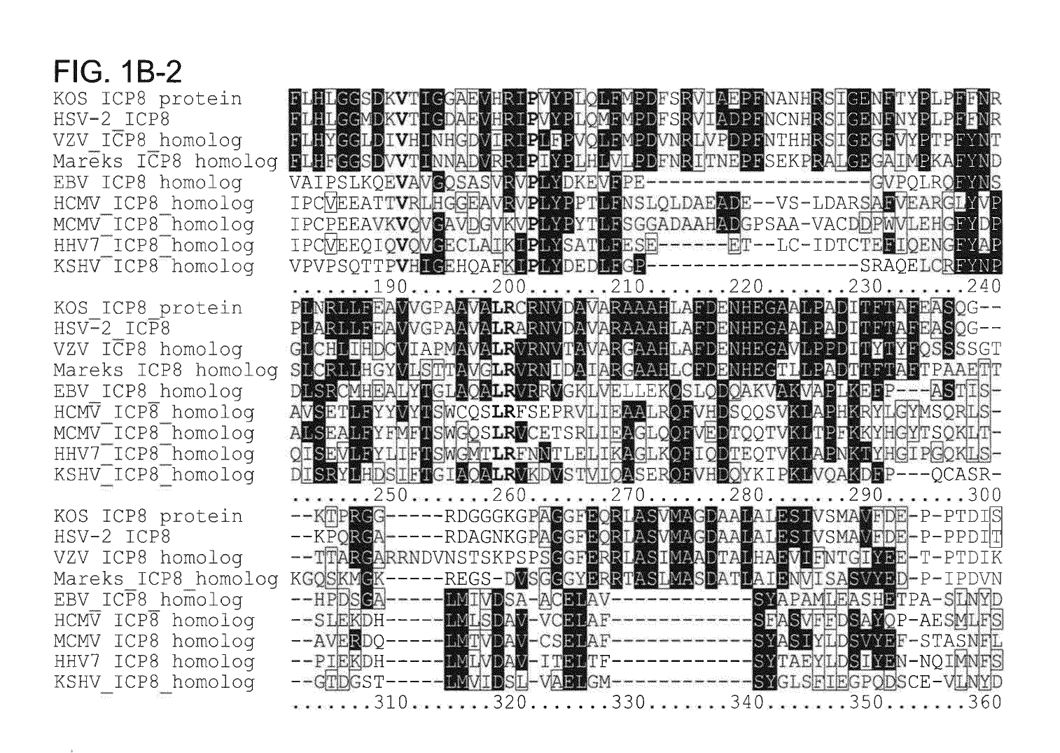

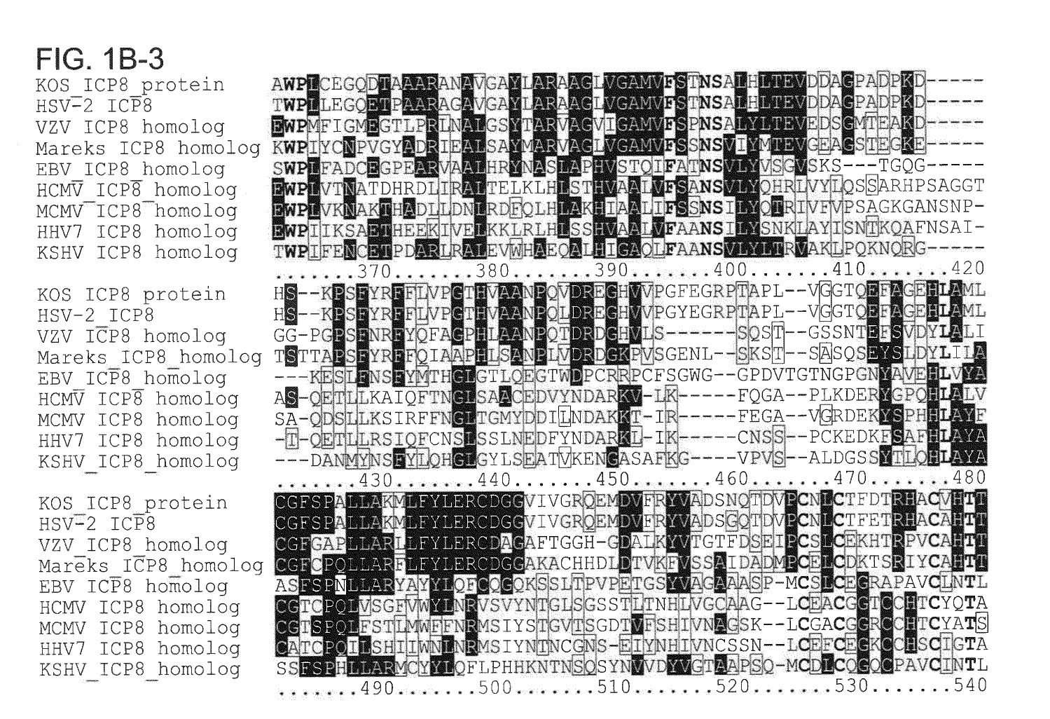

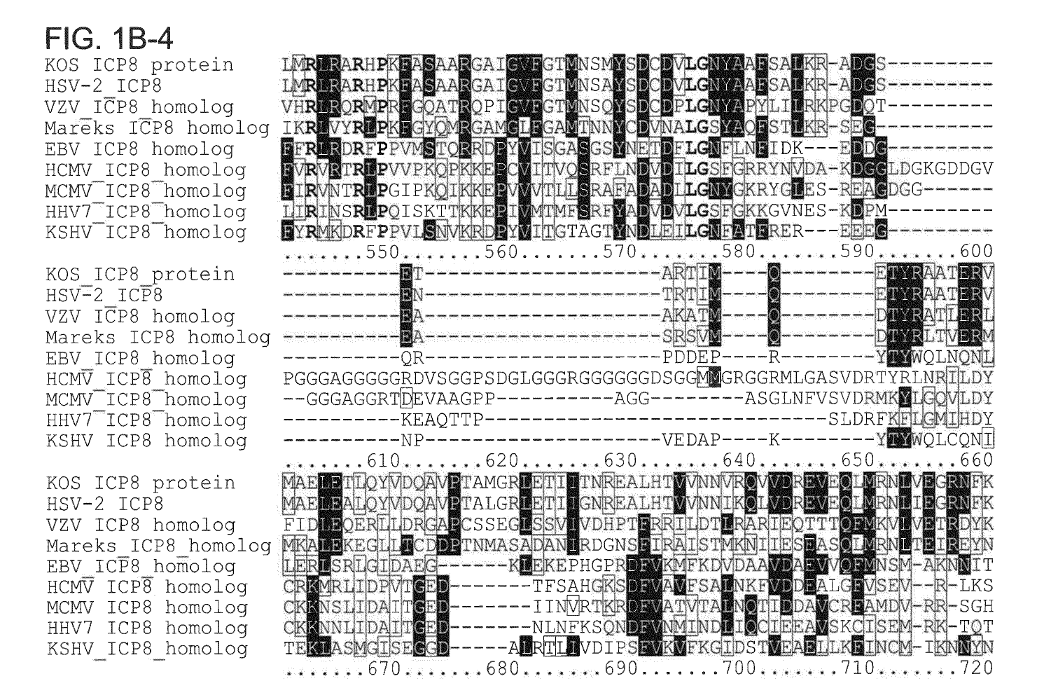

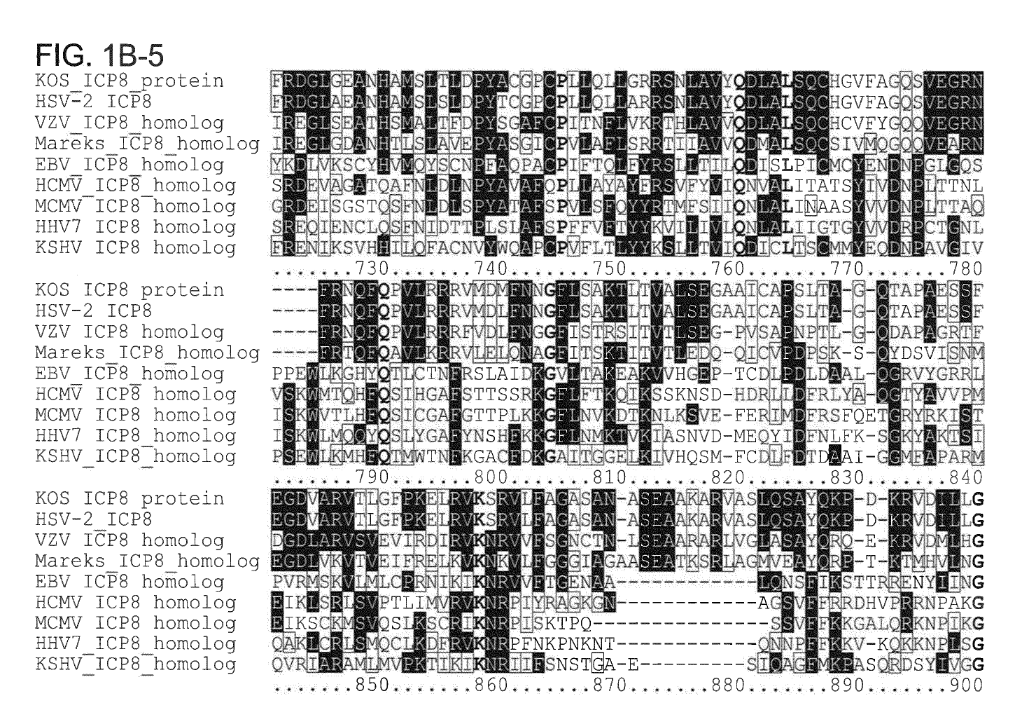

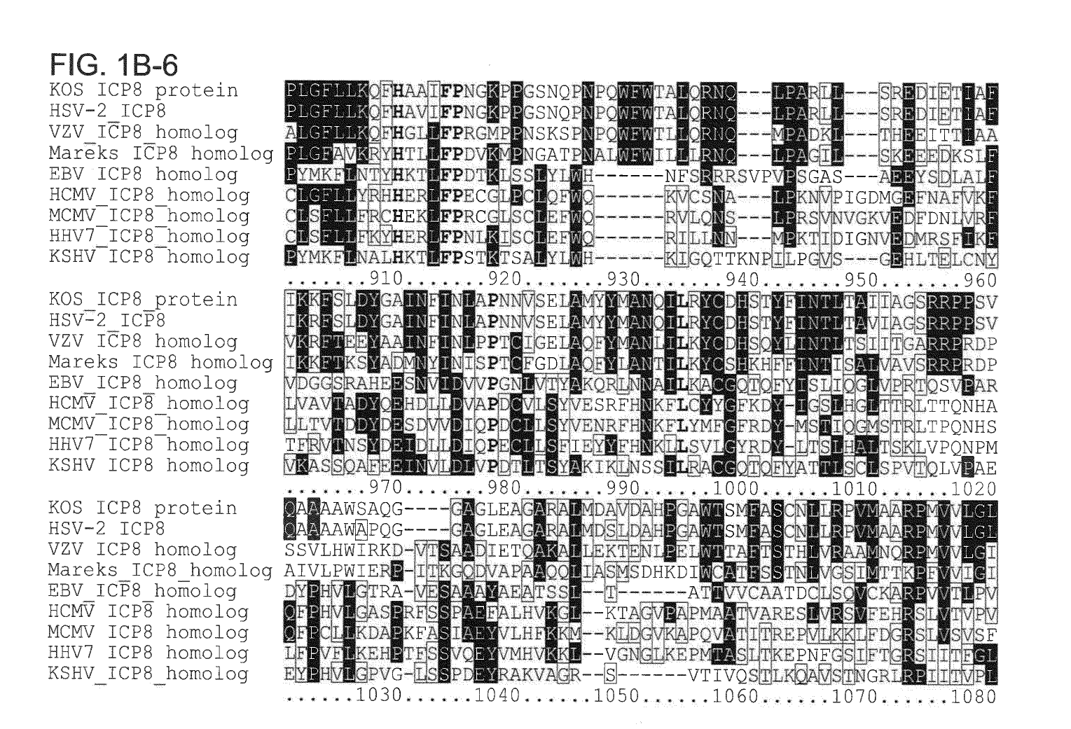

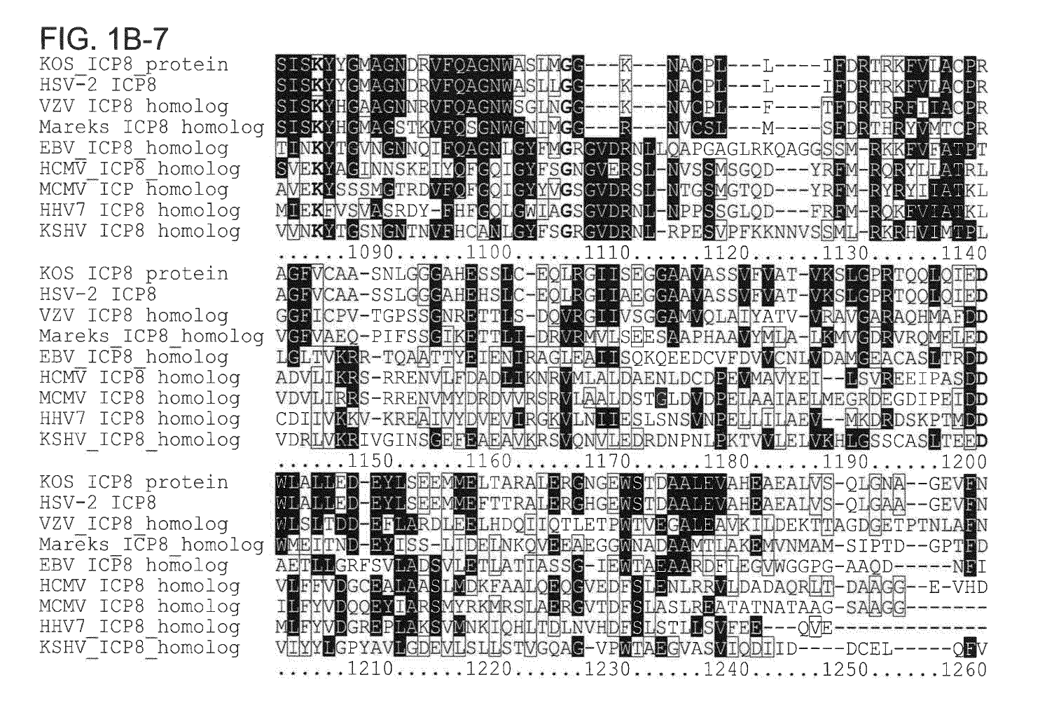

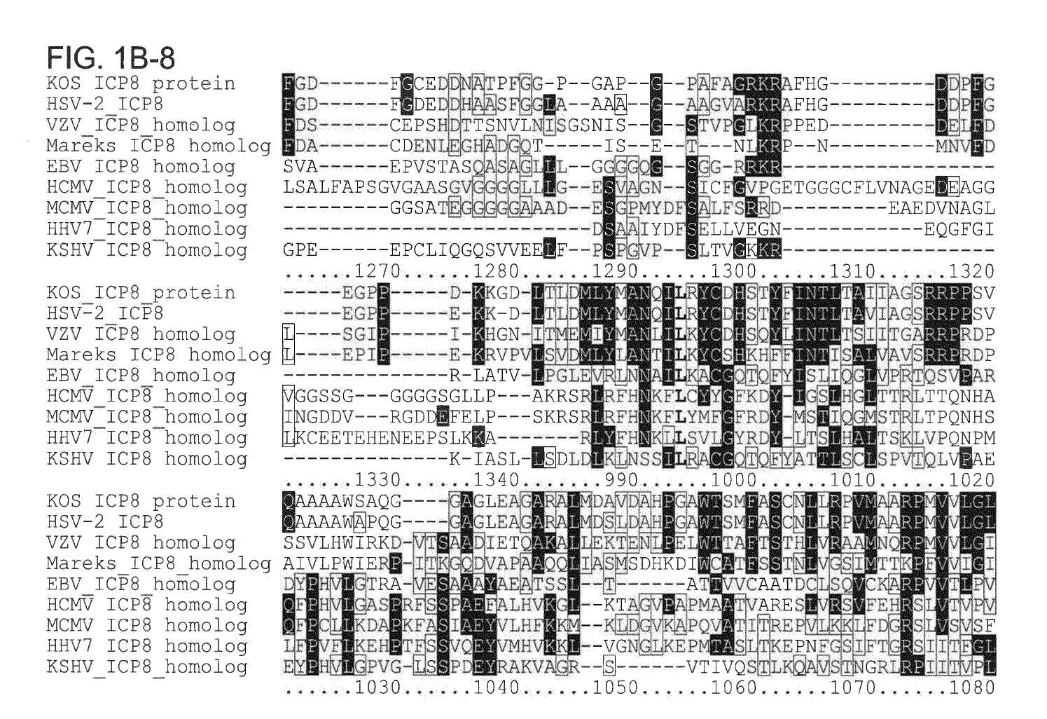

To identify highly conserved regions (and therefore new functional domains) in HSV-1 ICP8 and its homologs in other herpesviruses, an alignment of amino acid sequences from nine ICP8 homologs was performed, with representatives from alpha-, beta-, and gamma-herpesviruses. Numerous aspartic acid (D) and glutamic acid (E) residues were identified that were conserved in many or all of the ICP8 homologs (FIGS. 1A and 1B). The conservation of these residues in the homologs suggests that they are important for ICP8 function. Several of these conserved residues were located in or near the DNA binding groove in the ICP8 crystal structures (Mapelli, M., et al., J. Biol. Chem., vol. 280, pages 2990-2997), suggesting that they would be available to carry out enzymatic functions on bound DNA. Interestingly, members of a family of enzymes called DDE recombinases, including transposases, RAG-1, and retroviral integrases, also have conserved D and E residues that coordinate magnesium ions that are important for mediating the recombination reactions, leading to the notion that ICP8 may share biochemical and pharmacological properties with these will-studied proteins. Numerous ICP8 residues were further investigated, including D545 (amino acid positions are based on KOS ICP8 sequence)ₘ D547, D625, E627, D645, E735, E860, D861, E1086, and D1087.

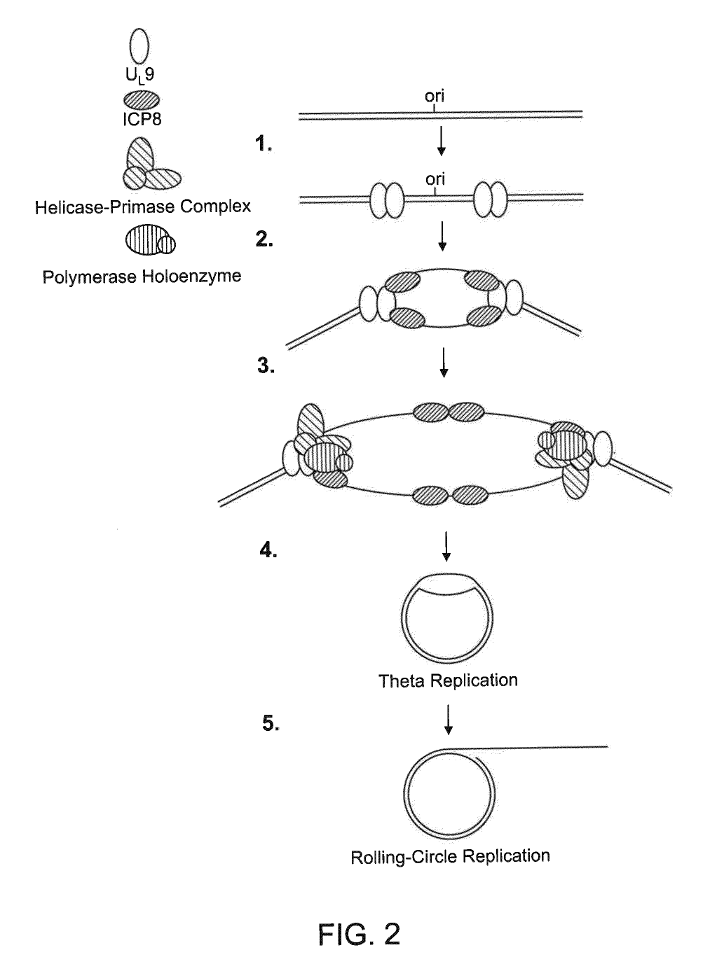

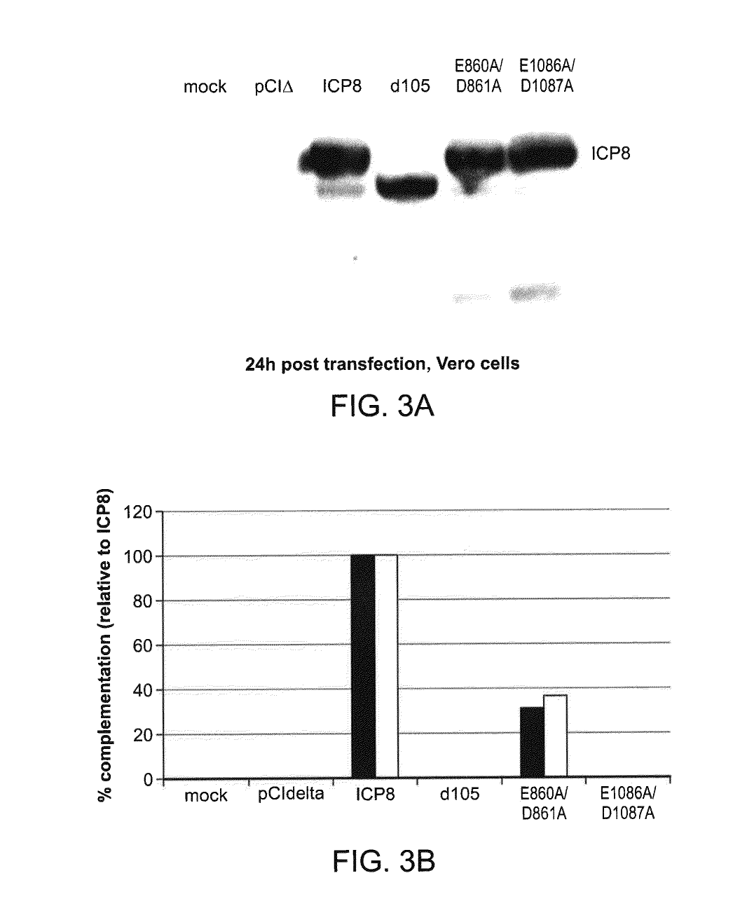

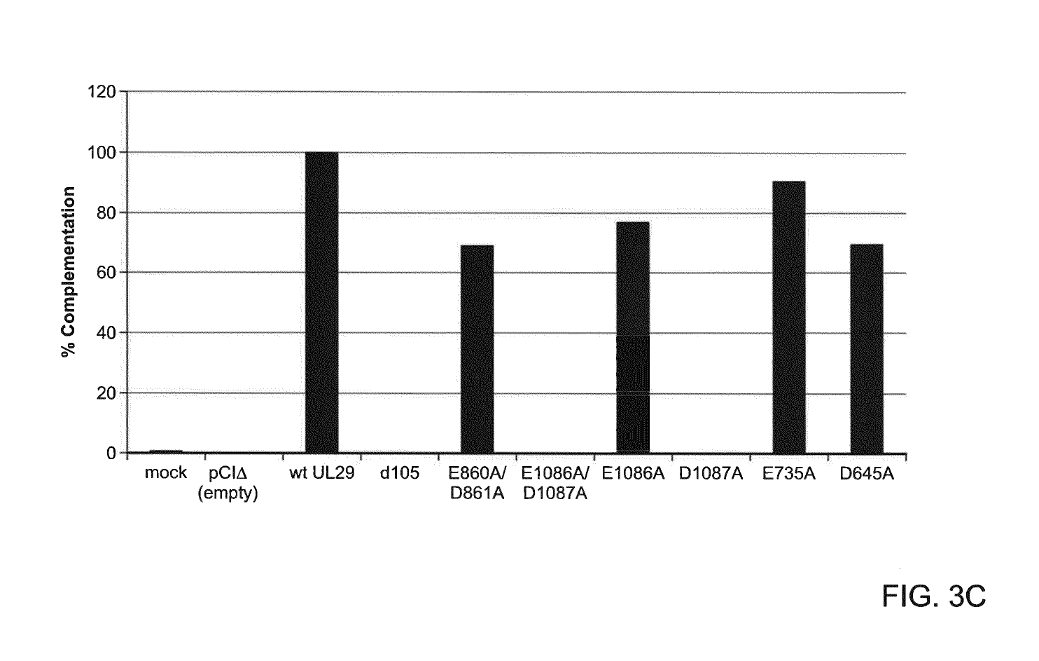

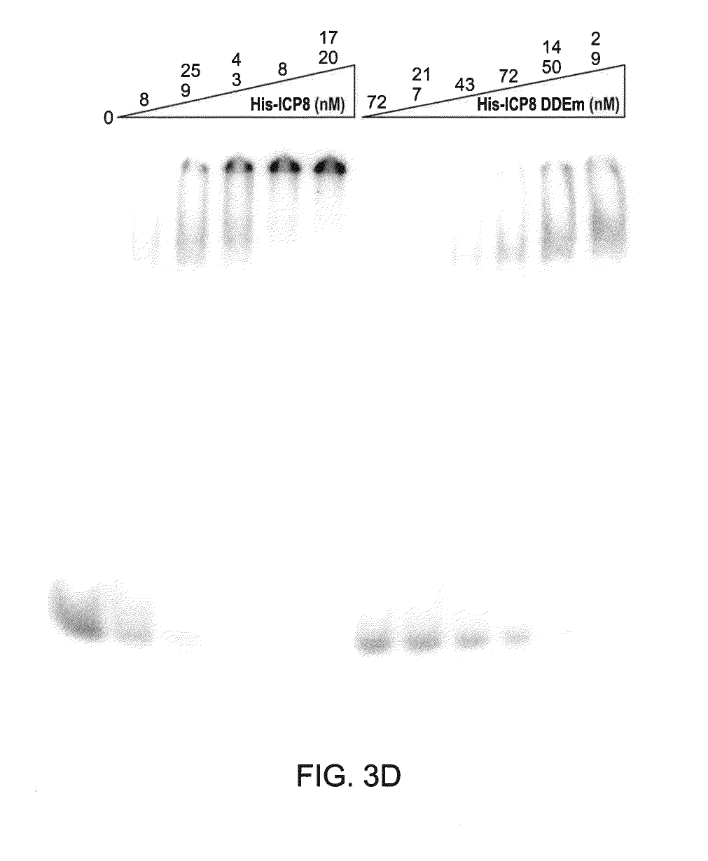

To determine whether the D643, E735, D860/E861 and E1086/D1087 DDE recombinase residues identified above are required for ICP8 function during HSV-1 infection, they were mutated to alanine and tested for their ability to complement the replication of an ICP8 mutant virus. FIG. 2 shows a schematic of the HSV-1 replication process. As shown in FIG. 3A, all mutant forms of ICP8 were expressed at similar levels in transiently transfected Vero cells. At 24 hours after transfection, Vero cells were infected with the ICP8 null virus 8lacZ at a multiplicity of infection (MOI) of 20. Samples were harvested at 24 hours post infection and the viral yield was determined by performing plaque assays on V529 cells, which stably express ICP8 to complement replication of 8lacZ. The viral yield observed in cells transfected with the plasmid expressing wild type ICP8 was designated as 100% complementation, and all of the ICP8 mutants were compared to that value. As shown in FIGS. 3B and 3C, the D860A/E861A mutant form of ICP8 complemented 8lacZ replication to approximately 37% the level of wild type ICP8, indicating that residues 860 and 861 are required for wild type activity of ICP8. The E1086A/D1087A mutant form of ICP8 did not complement replication of 8lacZ to above the background levels observed when cells were transfected with the empty vector plasmid, as shown in FIG. 3B. The d105 mutant form of ICP8, which fails to complement replication of an ICP8 mutant virus, also did not complement 8lacZ replication to above background levels. These results indicate that either residues 1086 and/or 1087 are very important for ICP8 function during HSV-1 replication.

To determine whether both residues 1086 and 1087 are required for ICP8 activity, each amino acid was mutated to alanine individually. FIG. 3C shows that E1086A displayed significant levels of complementation (˜60%), while D1087A displayed no detectable complementation, thereby indicating that D1087 plays a very important role in ICP8 function.

Additionally, mutations at two other locations in ICP8, E735 and D645, were also mutated to assess whether they were important for ICP8 function. As shown in FIG. 3C, E735A and D645A displayed complementation levels of ˜90% and ˜70%, respectively. This data indicates that these positions are less important for ICP8 function than position D1087.

To rule out that the possibility that mutation of the putative DDE residues in ICP8 did not reduce the activity of ICP8 by simply destroying the overall folding of the protein, we investigated their ability to bind DNA. As shown in FIG. 3D, shows that the DDE mutant is able to bind DNA, indicating that it possesses the requisite structure required to bind DNA. Additionally, Vero cells were either mock infected or infected with either wild type HSV-1 or the ICP8 DDE mutant at an MOI of 10. At 8 hours post infection, cells were fixed and stained for immunofluorescence with the ICP8-specific antibody 39S, which specifically recognizes active ICP8 in viral replication compartments. The DDE mutant of ICP8 was recognized by a conformation specific antibody (data not shown).

### Example 2

### The DDE Residues in ICP8 are Required for HSV-1 Replication and Viral DNA Replication

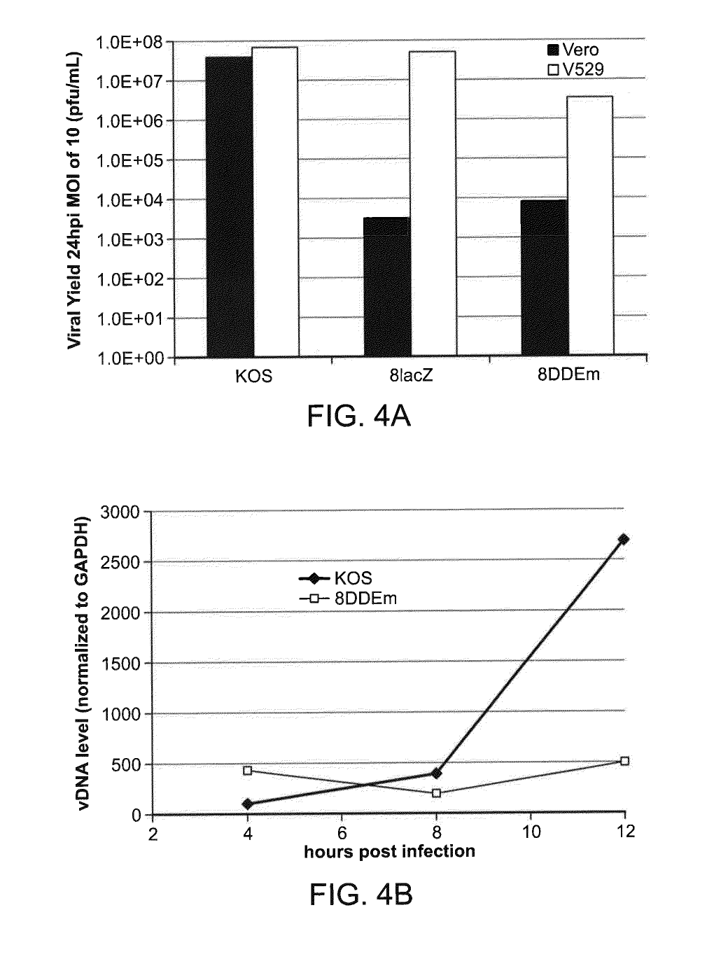

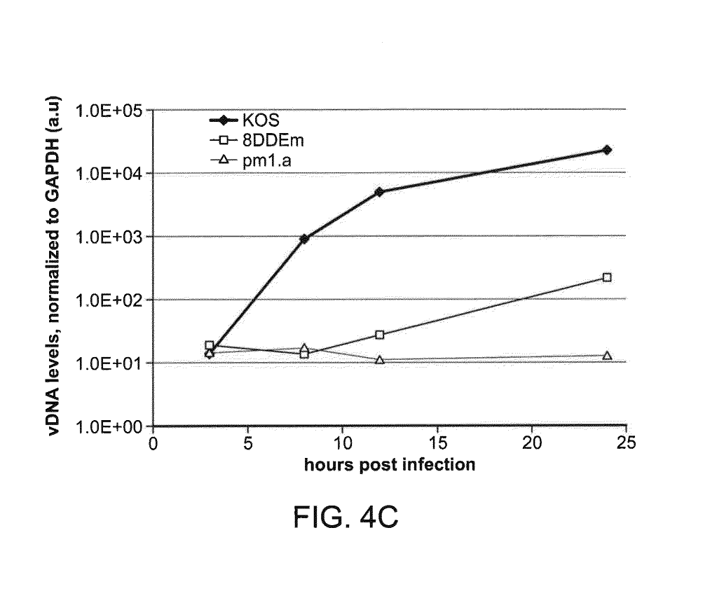

KOS.DDEm, a mutant virus containing the E1086A/D1087A mutation in ICP8, was constructed to investigate whether this mutant form of ICP8 affected HSV-1 replication when expressed from the viral genome. As shown in FIG. 4A, replication of this mutant virus was indistinguishable from the ICP8-null virus 8lacZ in non-complementing Vero cells. KOS.DDEm replicated to nearly wild type levels in the complementing V529 cells, suggesting that while this DDE mutation in ICP8 cannot support viral replication, it does not have a dominant negative phenotype, which is different from the d105 mutation.

The levels of viral DNA replication in Vero cells infected with either the KOS.DDEm mutant virus or wild type KOS were investigated. No viral DNA replication was observed between 4 and 12 hours post infection in cells infected with the KOS.DDEm mutant virus. In contrast, as shown in FIG. 4B, a more than 20-fold increase in HSV-1 DNA was observed by 12 hours post infection in cells infected with wild type virus. FIG. 4C further shows the effect of the KOS.DDEm mutant virus relative to another independent control, pm1.a., which is completely defective for DNA replication. These data indicate that the DDE residues in ICP8 are very important for HSV-1 DNA replication, and that these residues likely promote recombination activity on the viral genome.

### Example 3

### Effect of DDE Residues on Viral Gene Expression

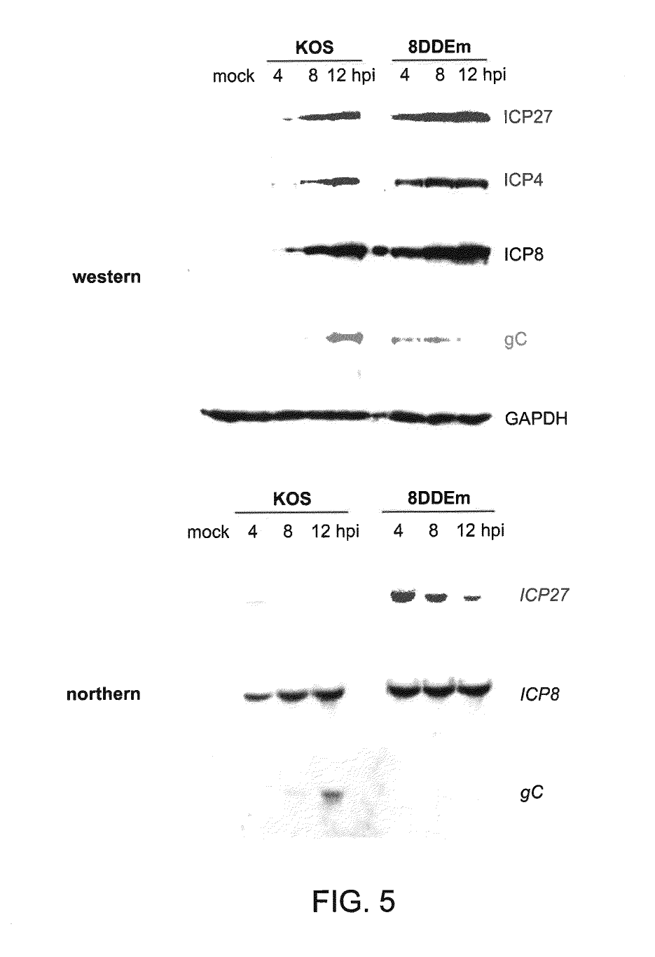

As described above, the KOS.DDEm mutant virus exhibited defects in viral replication and DNA replication; consequently, KOS.DDEm mutant virus was also tested for an effect on viral gene expression. The accumulation of the immediate-early gene products ICP27 and ICP4, the early gene product ICP8, and the late gene product glycoprotein C (gC), was assayed by performing immunoblot assays with Vero cells that were infected with either wild type HSV-1 or the ICP8 mutant virus KOS.DDEm. As shown in FIG. 5, slightly higher levels of ICP27, ICP4, and ICP8 were observed in KOS.DDEm-infected Vero cells, relative to Vero cells infected with wild type HSV-1, suggesting that the putative DDE recombinase residues in ICP8 are not required for expression of viral immediate-early or early genes. Although accumulation of the immediate-early and early gene products tested was not dependent on the DDE residues in ICP8, the late gene product gC was observed to accumulate to lower levels at 12 hours post infection in Vero cells infected with the KOS.DDEm relative to cells infected with wild type HSV-1, as shown in FIG. 5. Patterns of viral transcript accumulation observed in RNA hybridization assays were similar to the patterns of accumulation of viral proteins observed in immunoblot assays. It is known that expression of gC requires HSV-1 DNA replication, and the decreased levels of gC are consistent with the observed defect in viral DNA replication.

### Example 4

### Raltegravir, LL841411, and 118-D-24 Inhibit HSV-1 Replication with High Efficiency

The HIV integrase structurally similar to ICP8, and can be inhibited by specific drugs such as Raltegravir and 118-D-24. These drugs were tested to determine whether they could inhibit HSV replication.

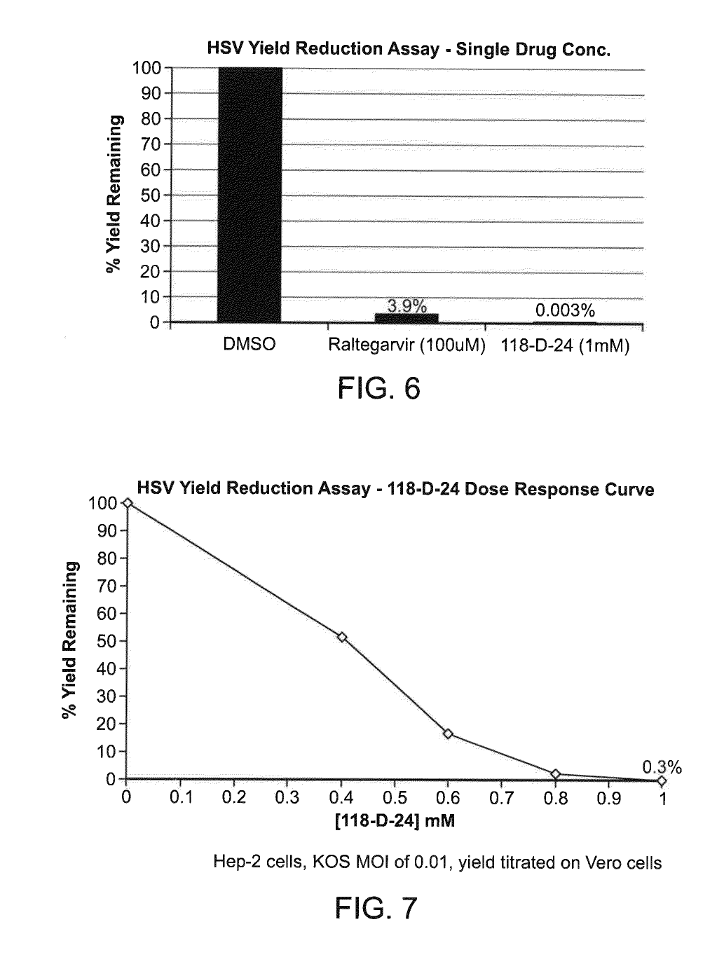

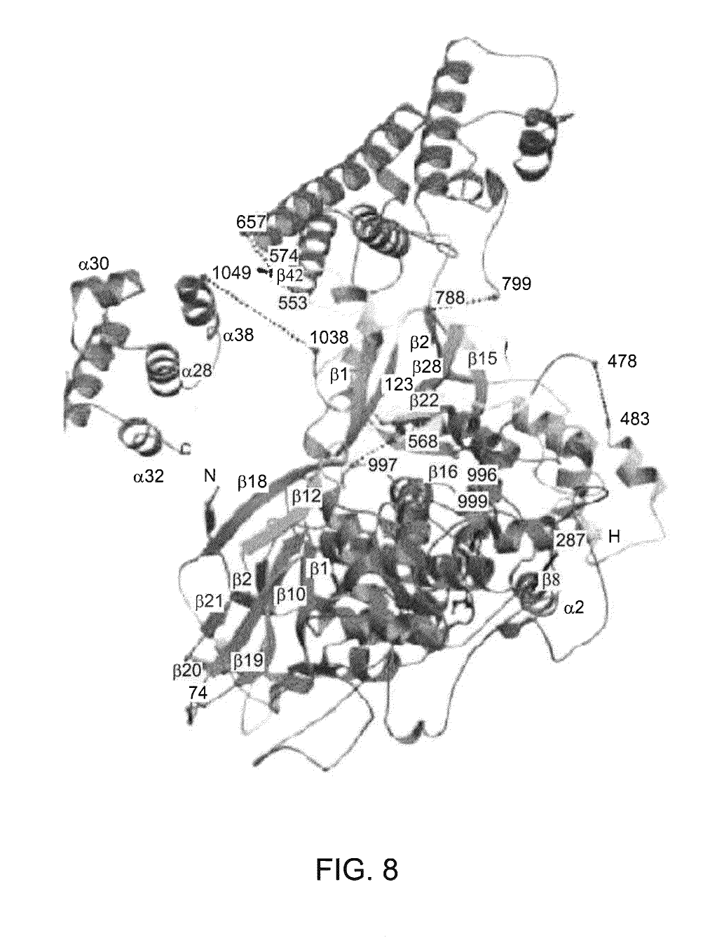

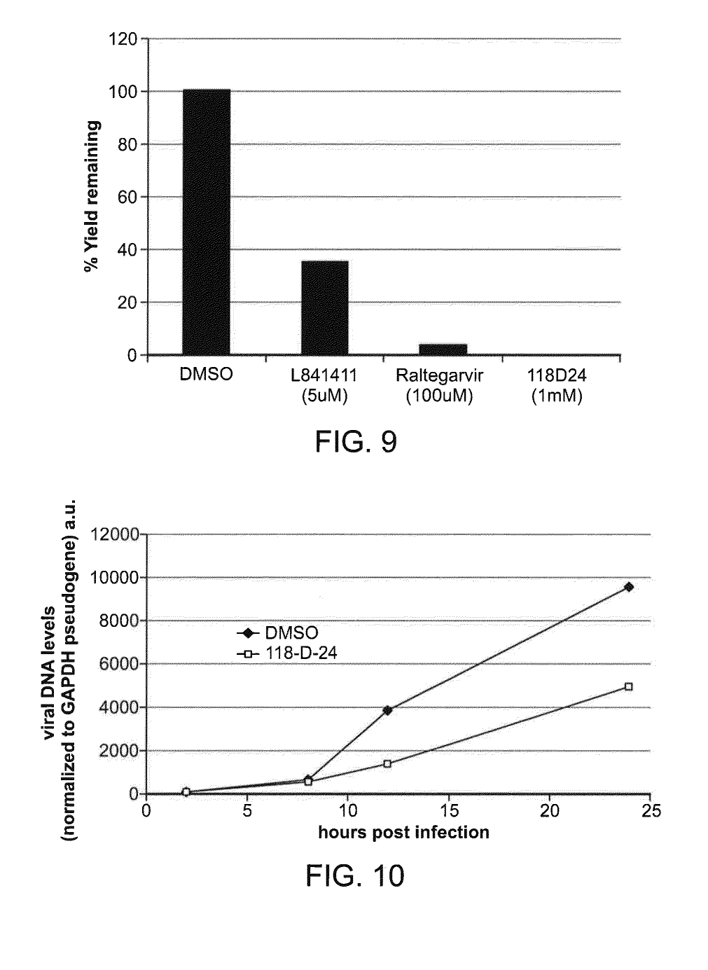

Raltegravir and 118-D-24, which have been shown to inhibit HIV replication by inhibiting the activity of the HIV integrase enzyme, inhibited the replication of HSV-1 with high efficacy in cell culture-based assays. As shown in FIG. 6, Raltegravir at a concentration of 100 μM reduced HSV viral yield by greater than 96%, and 118-D-24 at a concentration of 1 mM reduced HSV yield by greater than 99.99%. 118-D-24 was studied further because it appeared to inhibit HSV replication very strongly. As shown in FIG. 7, a dose-response curve with 118-D-24 demonstrated that the concentration required for 50% inhibition of HSV yield (IC50) was approximately 0.4 mM. The inhibition of HSV replication is likely due to the inhibition of the HSV protein ICP8, which shares structural homology with HIV integrase. FIG. 8 shows a ribbon structure of the ICP8 protein. Additionally, FIG. 9 shows that L-841411, another inhibitor, also shows significant reduction of HSV viral yield, albeit to a lesser extent than Raltegravir and 118-D-24.

### Example 5

### 118-D-24 Inhibit HSV-1 Replication with High Efficiency

In view of the high efficiency of 118-D-24 as an HSV replication inhibitor, the effective concentration range of 118-D-24 was tested over a concentration range of 0-1 mM. FIG. 7 shows the dose response curve of 118-D-24 as tested in an HSV yield reduction assay. 118-D-24 reduces HSV yield by about 50% at a concentration of 0.4 mM, and completely, or nearly completely, eliminates HSV yield at a concentration of 1 mM.

The effect of 118-D-24 on viral DNA replication was also tested. As shown in FIG. 10, 118-D-24 decreased viral DNA replication by about 50% relative to a DMSO control.

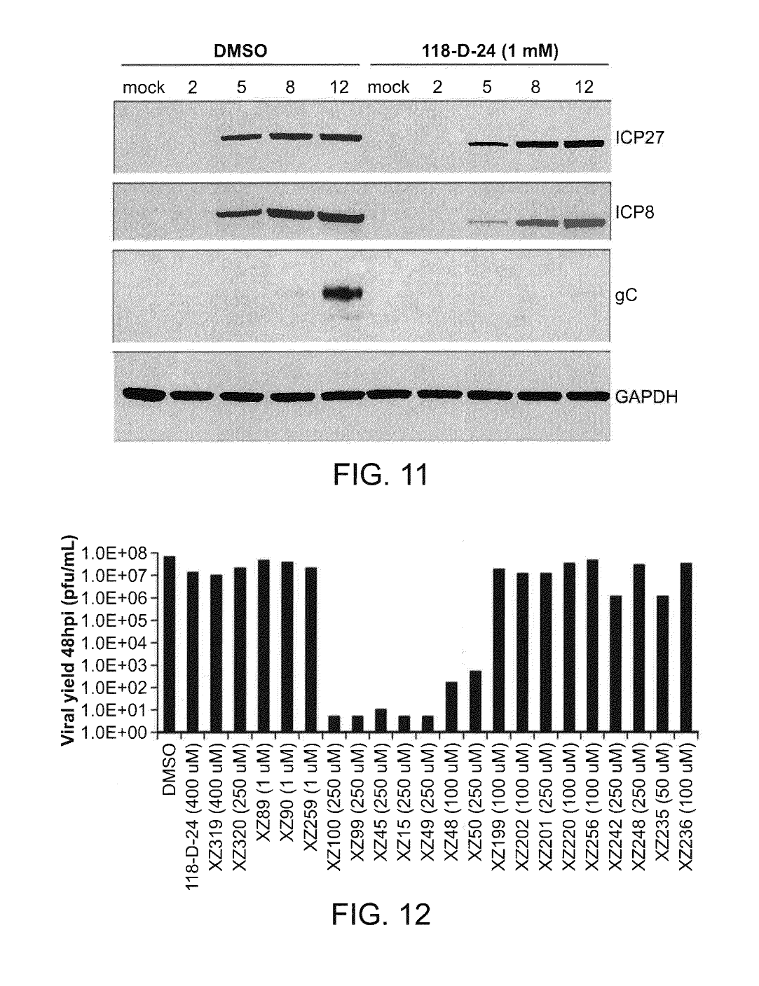

The effect of 118-D-24 on viral gene expression was also tested. As shown in FIG. 11, the accumulation of the immediate-early gene product ICP27, and the early gene product ICP8, was moderately reduced at 5 hours post-treatment relative to a DMSO control, however, accumulation of the late gene product gC was eliminated.

### Example 6

### 118-D-24 Derivatives Inhibit HSV Replication

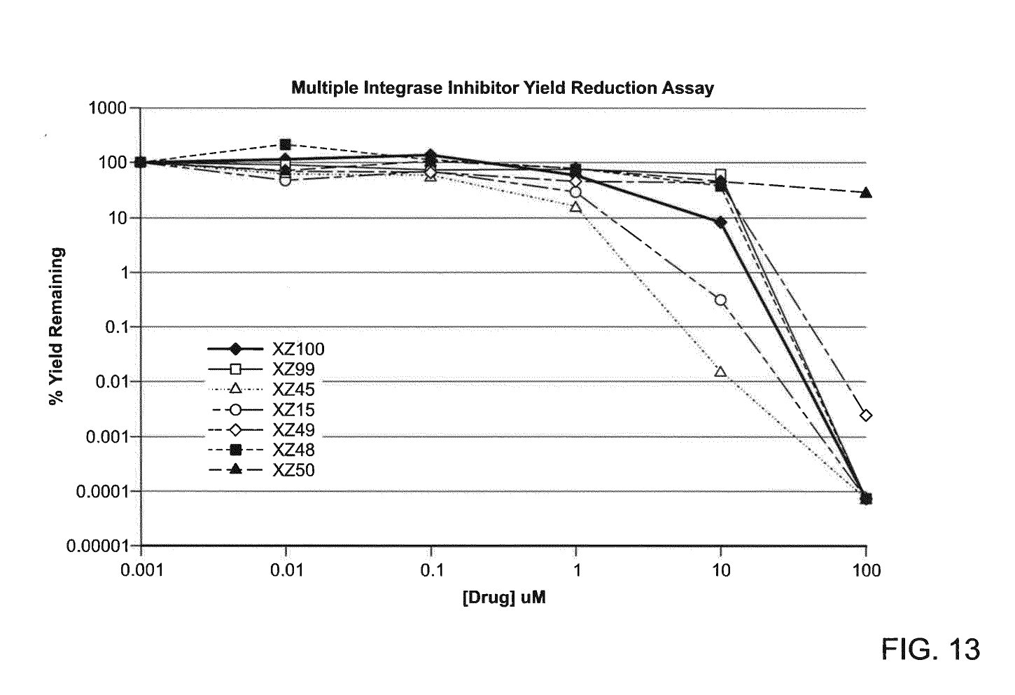

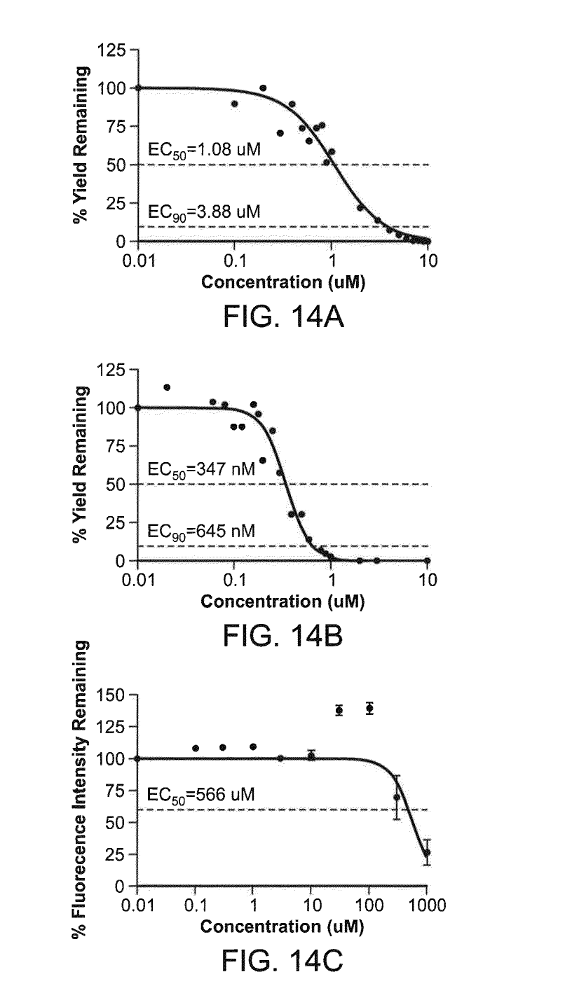

To evaluate the effects of 118-D-24 derivatives on HSV replication, a panel of derivatives (Table 1) (Zhao, X. Z., 2008, J. Med. Chem., vol. 51, pages 251-259) were screened for their ability to inhibit HSV-1 KOS virus replication in Hep2 cells. Hep2 cells were plated in a 6-well plate and incubated overnight to reach confluency. The cells were then inoculated with HSV-1 KOS virus at MOI=0.01 for 1 hour. Following inoculation, the virus inoculum was replaced with DMEV medium containing 118-D-24 or a derivative of 118-D-24. After 48 hours, samples were harvested by adding an equal volume of 10% non-fat milk and immediately frozen at −80° C. Samples were freeze-thawed three times to rupture the cell membranes and allow the release of virus. Virus titers were determined by titration on Vero cells. The 100% yield represents the viral titer in samples without drug treatment. As shown in FIG. 12, 250 μM of the 118-D-24 derivatives XZ100, XZ99, XZ45, XZ15, XZ49, XZ48, and XZ50 resulted in significant reductions in viral yield. Dose response curves for these 118-D-24 derivatives on viral replication are shown in FIG. 13. Hep2 cells or (FIG. 14A) or HFF cells (FIG. 14B) were infected with HSV-1 virus (KOS strain) at MOI of 0.01. Samples were then processed and percent yield remaining values were calculated. EC50 and EC90 values were calculated using nonlinear regression curve fit with a variable slope. GaraphPad Prism 5 software was used for all analyses. (FIG. 14C) Cytotoxicity of XZ45 on the viability of Hep2 cells during a 48-hour incubation period was evaluated using the Promega cell titer glo assay, as described by the manufacturer. The reported values are percent fluorescence intensity remaining relative to the fluorescence intensity from cells grown in media containing DMSO alone. CC50 value was determined as described above. FIGS. 14A and 14B show the effects of XZ45 on HSV-1 replication in Hep2 cells (14A) and normal human foreskin fibroblasts (14B) and the cytotoxicity of XZ45 in Hep2 cells is shown in FIG. 14C. These results indicate that XZ45 has a therapeutic index of ˜500 for HSV-1 in the Hep2 cell system.

TABLE 1
118-D-24 derivatives

| NO | STRUCTURE |
|---|---|
| XZ319 | 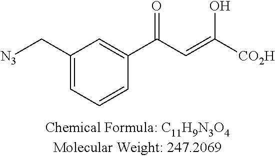 [SMILES: [N-]=[N+]=NCc1cccc(C(=O)/C=C(\O)C(=O)O)c1] |
| XZ320 | 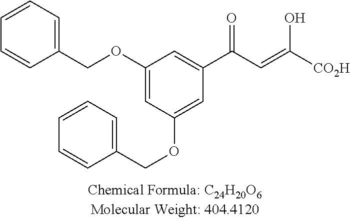 [SMILES: O=C(O)/C(O)=C/C(=O)c1cc(OCc2ccccc2)cc(OCc2ccccc2)c1] |
| XZ89 | 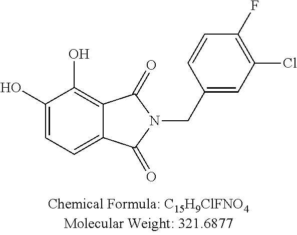 [SMILES: O=C1c2ccc(O)c(O)c2C(=O)N1Cc1ccc(F)c(Cl)c1] |
| XZ90 | 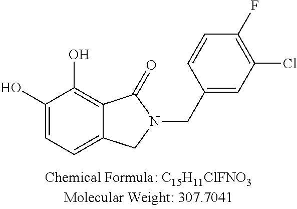 [SMILES: O=C1c2c(ccc(O)c2O)CN1Cc1ccc(F)c(Cl)c1] |
| XZ259 | 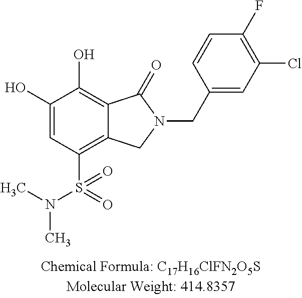 [SMILES: CN(C)S(=O)(=O)c1cc(O)c(O)c2c1CN(Cc1ccc(F)c(Cl)c1)C2=O] |
| XZ100 | 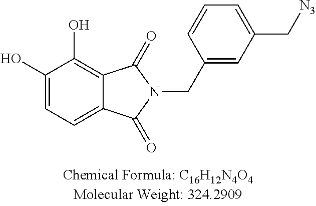 [SMILES: [N-]=[N+]=NCc1cccc(CN2C(=O)c3ccc(O)c(O)c3C2=O)c1] |
| XZ99 | 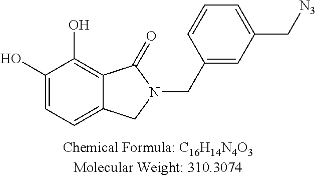 [SMILES: [N-]=[N+]=NCc1cccc(CN2Cc3ccc(O)c(O)c3C2=O)c1] |
| XZ45 | 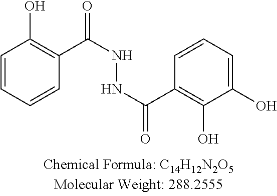 [SMILES: O=C(NNC(=O)c1cccc(O)c1O)c1ccccc1O] |
| XZ15 | 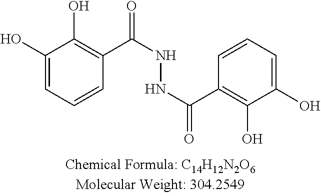 [SMILES: O=C(NNC(=O)c1cccc(O)c1O)c1cccc(O)c1O] |
| XZ49 | 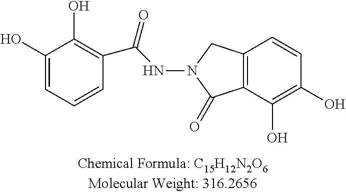 [SMILES: O=C(NN1Cc2ccc(O)c(O)c2C1=O)c1cccc(O)c1O] |
| XZ48 | 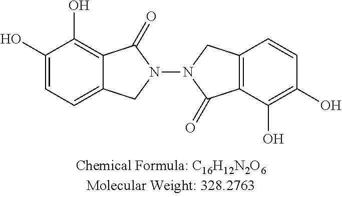 [SMILES: O=C1c2c(ccc(O)c2O)CN1N1Cc2ccc(O)c(O)c2C1=O] |
| XZ50 | 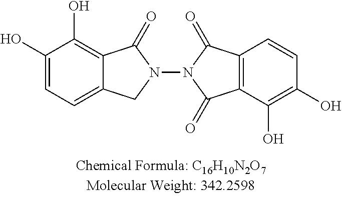 [SMILES: O=C1c2c(ccc(O)c2O)CN1N1C(=O)c2ccc(O)c(O)c2C1=O] |
| XZ199 | 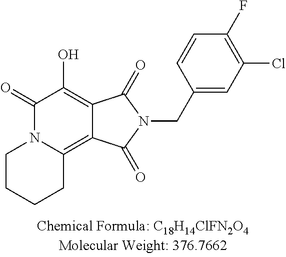 [SMILES: O=C1c2c(c3n(c(=O)c2O)CCCC3)C(=O)N1Cc1ccc(F)c(Cl)c1] |
| XZ202 | 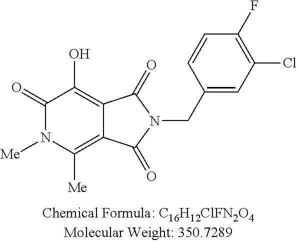 [SMILES: Cc1c2c(c(O)c(=O)n1C)C(=O)N(Cc1ccc(F)c(Cl)c1)C2=O] |
| XZ201 | 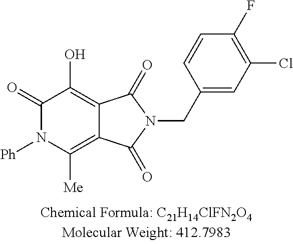 [SMILES: Cc1c2c(c(O)c(=O)n1-c1ccccc1)C(=O)N(Cc1ccc(F)c(Cl)c1)C2=O] |
| XZ220 | 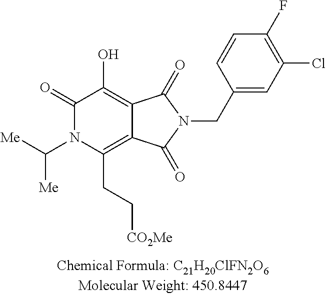 [SMILES: COC(=O)CCc1c2c(c(O)c(=O)n1C(C)C)C(=O)N(Cc1ccc(F)c(Cl)c1)C2=O] |
| XZ256 | 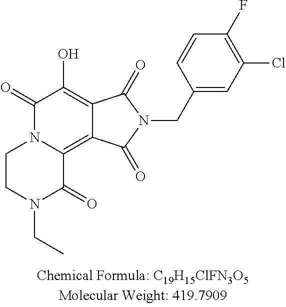 [SMILES: CCN1CCn2c(c3c(c(O)c2=O)C(=O)N(Cc2ccc(F)c(Cl)c2)C3=O)C1=O] |
| XZ242 | 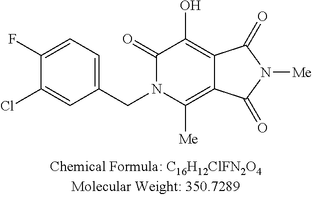 [SMILES: Cc1c2c(c(O)c(=O)n1Cc1ccc(F)c(Cl)c1)C(=O)N(C)C2=O] |
| XZ248 | 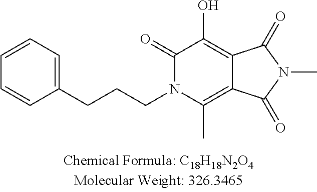 [SMILES: Cc1c2c(c(O)c(=O)n1CCCc1ccccc1)C(=O)N(C)C2=O] |
| XZ235 | 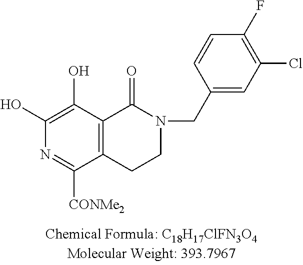 |
| XZ236 | 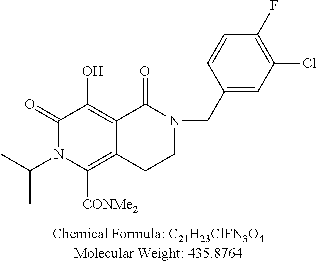 |

### Example 7

### XZ45 Inhibits the Replication of HSV-1, HSV-2, and Human Cytomegalovirus (HCMV)

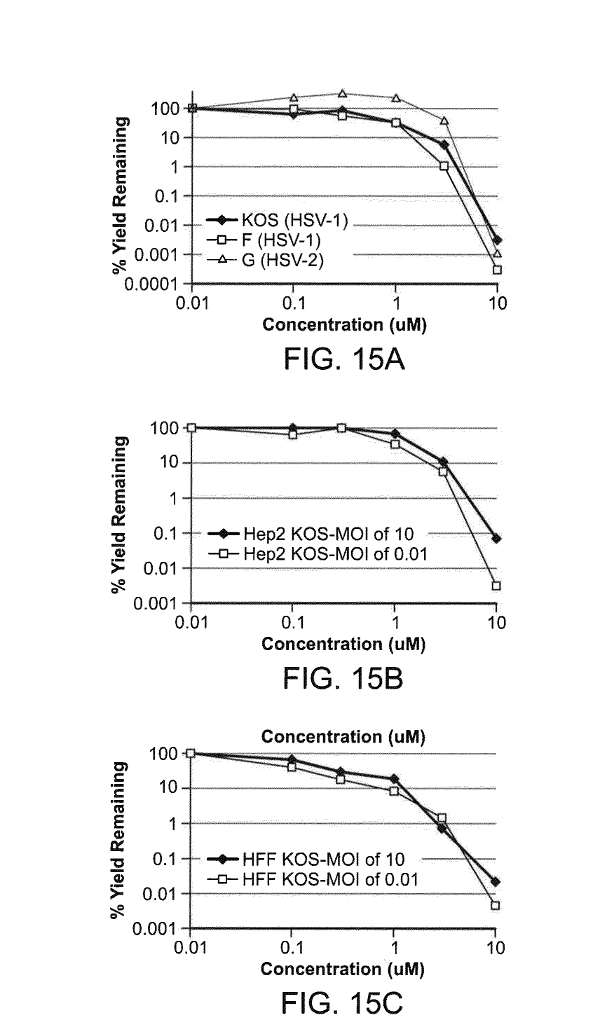

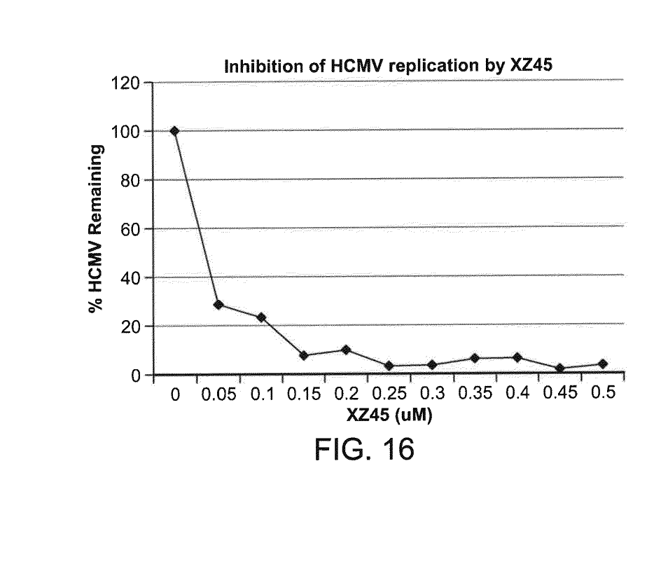

Hep2 cells were infected with HSV-1 strain KOS, strain F, or HSV-2 strain G at MOI of 0.01. The infected cells were grown in media containing increasing concentrations of XZ45 or DMSO for 48 hours. Samples were harvested and viral yield was determined by plaque assay on Vero cells. The reported values are percent yield remaining relative to cells grown in media containing DMSO alone (FIG. 15A). Hep2 cells (FIG. 15B) or HFF cells (FIG. 15C) were infected with HSV-1 virus (KOS strain) at MOI of 10 or 0.01 or with human cytomegalovirus (HCMV) (FIG. 16). Samples were then processed and percent yield remaining values were calculated as describe above. The results demonstrate that XZ45 inhibits the replication of HSV-1, HSV-2, and HCMV.

### Example 8

### XZ45 Inhibits Viral DNA Synthesis and Late Gene Expression

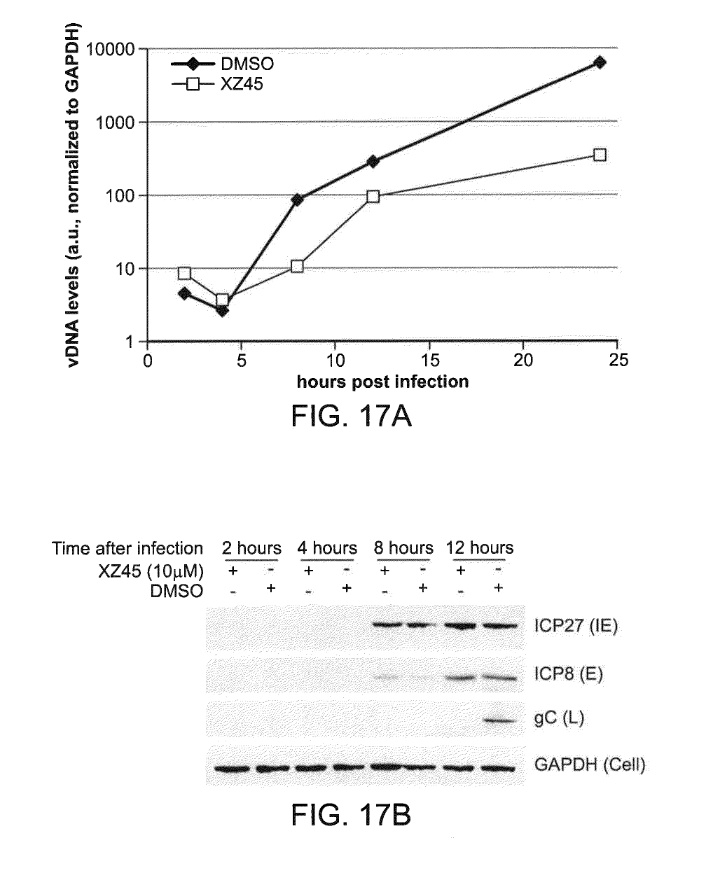

FIG. 17A shows the effect of XZ45 on viral DNA synthesis. Hep2 cells were infected with HSV-1 KOS at an MOI=10 in the presence or absence of 10 μM XZ45. Total DNA was harvested at the times indicated, and viral DNA levels in each sample were determined by real-time PCR and were normalized to the levels of cellular DNA. FIG. 17B shows the effect of XZ45 on viral gene expression. Hep2 cells were infected with HSV-1 KOS in the presence or absence of 10 μM XZ45. Lysates were prepared for immunoblotting at the indicated times. Polypeptides were resolved by SDS-PAGE, transferred to a PVDF membrane, and probed for representative immediate-early (ICP27), early (ICP8), and late (gC) gene products. As shown, XZ45 significantly inhibited the expression of the late gene (gC) products.

### Example 9

### XZ45 does not Decrease ssDNA Binding by ICP8

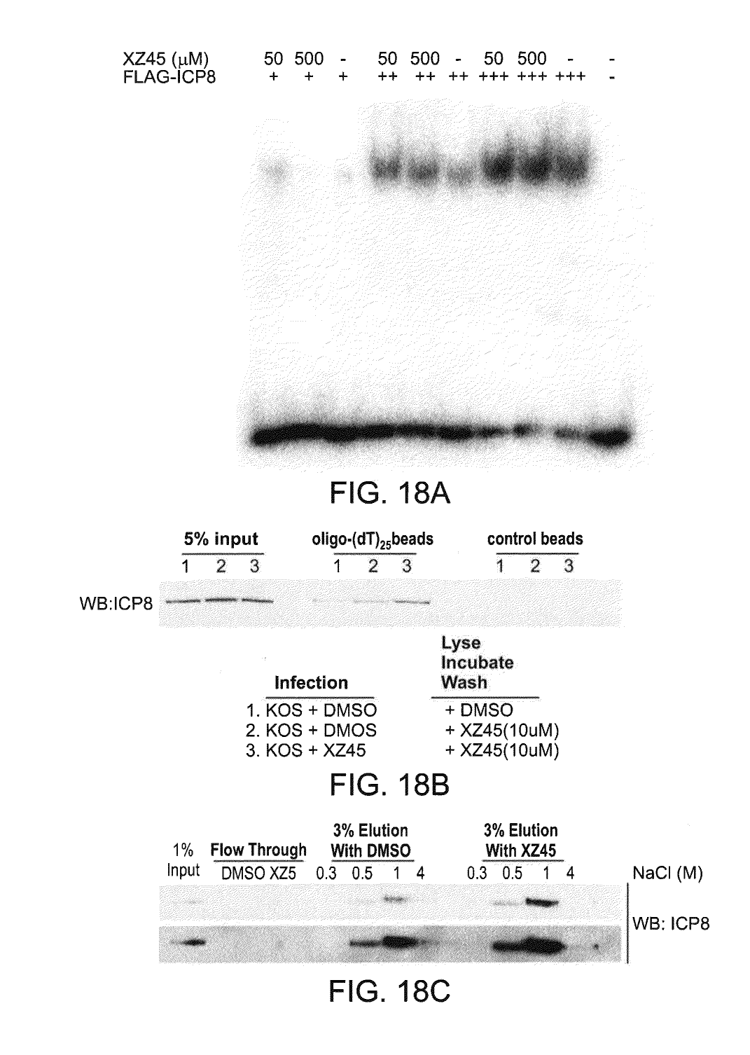

The effect of XZ45 on ssDNA binding by ICP8 was measured by EMSA assay with purified ICP8 protein using ³²P-labled polynucleotide ssDNA probe (FIG. 18A); ssDNA beads pull down assay (FIG. 18B), and ICP8 binding to ssDNA-cellulose (FIG. 18C). As shown, XZ45 does not affect ssDNA binding by ICP8 as determined using the three assays.

### Example 10

### XZ45 Inhibits Viral Recombination in Infected Cells

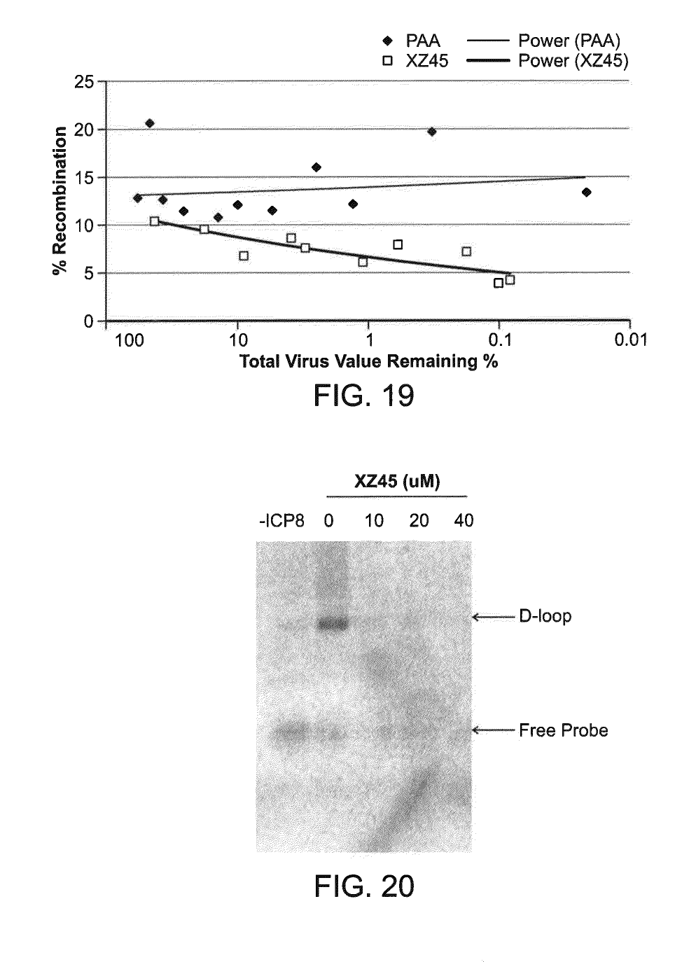

To test the effect of XZ45 on HSV recombination during viral replication, 8LacZ (deletion of ul29) and hr99 (deletion of ul5) virus were used to coinfect Hep2 cells in the presence of XZ45 or PAA. At 20 hours after infection, samples were harvested and progeny virus were tittered on Vero cells and V529 cells to determine the viral titer of recombinated virus and total virus. The recombination rate reflects the ratio between the titer of recombinated virus and total virus. As shown in FIG. 19, XZ45 significantly inhibited HSV recombination compared to PAA.

To further test the effect of XZ45 on ICP8 mediated recombination a D-loop assay was used. A double stranded DNA probe was mixed with a single stranded DNA oligonucleotide in the presence or absence of ICP8 with 0, 10, 20, or 40 μM XZ45. Following incubation, the reaction products were analyzed by electrophoresis through a native gel. As shown in FIG. 20, ICP8 is able to catalyze recombination between a double stranded DNA probe and a single stranded DNA oligonucleotide. However, XZ45 inhibited the ICP8 mediated formation of D-loops between the double stranded DNA template and the single stranded DNA probe, thereby demonstrating that XZ45 inhibited the ability of ICP8 to mediate recombination.

The results described above were obtained using the following methods and materials.

Cells and Viruses.

Vero cells were obtained from American Type Cell Culture (Manassas, Va.). V529 cells were generated as described by, hereby incorporated by reference in its entirety. Cells were maintained in Dulbecco's Modification of Eagle's Medium (DMEM) supplemented with 5% heat-inactivated fetal bovine serum and 5% heat-inactivated newborn calf serum (NCS). Medium for the V529 cells was also supplemented with 500 μg/mL G418.

All experiments were performed with HSV-1 wild type strain KOS or mutant viruses 8lacZ, hereby incorporated by reference in its entirety) and KOS.8DDEm, which were derived from strain KOS. Viruses were propagated and titrated on Vero or V529 cells following standard procedures.

Plasmids.

The DDE mutations in ICP8 were generated by performing PCR-based site-directed mutagenesis.

Complementation Assay.

Vero cells, which do not complement the replication of the ICP8 mutant virus 8lacZ, were transfected with the indicated plasmid using standard transfection reagents known in the art (e.g. Effectene transfection reagents (Qiagen) according to the manufacturer's instructions). At 24 hours post transfection, the transfected cells were infected with 8lacZ at an MOI of 10 pfu/cell. At 24 hours post infection, viral yield samples were harvested by scraping the infected cell monolayer and collecting both the cells and the supernatant. Samples were frozen at −80° C., thawed, and cell-free supernatant was collected following centrifugation of the samples. Viral yield in each sample was determined by performing plaque assays on V529 cells, which express ICP8 and thus complement replication of the ICP8 mutant 8lacZ. Complementation was compared to the viral yield seen following transfection with the plasmid expressing wild type ICP8, and this value was set to be 100% complementation.

Construction of Mutant Viruses.

The plasmid p8-8GFP, which encodes ICP8 fused to GFP at its C terminus (described above), was linearized by digesting with EcoRI co-transfected into V529 cells with HSV-1 strain KOS infectious DNA, which was prepared using standard methods, to generate the recombinant virus KOS.8GFP. Plaques expressing GFP were identified by fluorescence microscopy and these recombinant viruses were plaque purified at least 3 times prior to use in experiments. To generate KOS.8DDEm, KOS.8GFP infectious DNA was co-transfected into V529 cells together with EcoRI linearized pBS.8flank8. Plaques that did not express GFP were identified by fluorescent microscopy and plaque purified 3 times prior to use in experiments. The presence of the DDE mutation in ICP8 was confirmed by sequencing a PCR product from the appropriate region of ICP8.

Viral Replication Assay.

Vero or V529 cells were infected with the indicated virus at an MOI of 10 in phosphate-buffered saline supplemented with calcium and magnesium (PBS-ABC) containing 1% FBS and 0.1% glucose for one hour in a shaking incubator at 37° C. Following the one hour adsorption step, cells were washed twice with acid wash buffer (recipe), once with DMEM containing 1% FBS, and then DMEM containing 1% FBS was added. Viral yields were harvested at the indicated time post infection by scraping the infected cell monolayer and collecting the cells and supernatant. Samples were frozen at −80° C. following harvesting. Viral yield was determined by performing plaque assays on Vero or V529 cells, as indicated.

Viral DNA Replication Assay.

Vero or V529 cells were infected with the indicated virus as described above for the viral replication assay. Following infection for the indicated time, samples were harvested by washing the cell monolayers with PBS-ABC, the cells were scraped in PBS-ABC, and the cells were then collected by centrifugation. Total DNA (including both cellular and viral DNA) was purified using standard methods (e.g. the Generation Capture Column Kit (Qiagen), according to the manufacturer's instructions). Viral DNA was quantified by performing real time PCR using primers specific for the ICP27 promoter. The Real time PCR was performed using standard reagents and methods known in the art (e.g. PowerSYBR Green reagents (Applied Biosystems) and an Applied Biosystems 7X00 Sequence Detection System, according to the manufacturer's instructions). The viral DNA levels were normalized to the levels of a GAPDH pseudogene in each sample.

Immunoblotting.

Vero or V529 cells were infected with the indicated virus as described above. Cell monolayers were washed with PBS-ABC, and lysates were prepared by scraping the cells in 2×SDS-PAGE loading buffer and boiling for 5 minutes. Polypeptides were resolved by SDS-PAGE and transferred to a polyvinylidene difluoride (PVDF) membrane. Membranes were blocked for 1 hour at room temperature with 5% milk in Tris-buffered saline with 0.1% Tween 20 (TBST). Blocked membranes were reacted with primary antibodies diluted in 5% milk in TBST.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of inhibiting herpes virus replication in a cell, the method comprising contacting the cell with an agent that inhibits a DDE recombinase, thereby inhibiting herpes virus replication in the cell.

2. A method of inhibiting herpes virus replication in a cell, the method comprising contacting the cell with an agent that reduces the biological activity of a herpes virus polypeptide having functional and/or structural homology to a human immunodeficiency virus (HIV) integrase, thereby inhibiting herpes virus replication in the cell.

3. The method of claim 2, wherein the polypeptide is ICP8.

4. The method of claim 1, wherein the agent is a small compound that inhibits Human Immunodeficiency Virus (HIV) integrase enzymatic activity.

5. The method of claim 1, wherein the agent is selected from the group consisting of Raltegravir, 118-D-24, L-841411, elvitegrevir, MK-2048, XZ100, XZ99, XZ45, XZ15, XZ49, XZ48, and XZ50; or a derivative or analog thereof.

6. (canceled)

7. (canceled)

8. A method of treating or preventing a herpes virus infection in a subject, the method comprising administering to the subject an effective amount of an agent that inhibits a DDE recombinase, or administering to the subject an effective amount of an agent that reduces the biological activity of a herpes virus polypeptide having functional and/or structural homology to a human immunodeficiency virus (HIV) integrase, thereby treating or preventing a herpes virus infection in the subject.

9. (canceled)

10. The method of claim 8, wherein the agent reduces herpes virus replication.

11. (canceled)

12. The method of claim 8, wherein the agent is selected from the group consisting of Raltegravir, 118-D-24, L-841411, elvitegrevir, MK-2048, XZ100, XZ99, XZ45, XZ15, XZ49, XZ48, and XZ50; or a derivative or analog thereof.

13. (canceled)

14. (canceled)

15. The method of claim 8, wherein the method further comprises identifying the subject as having or at risk of developing a herpes virus infection.

16. The method of claim 8, wherein the method further comprises identifying the subject as testing negative for an HIV infection.

17. The method of claim 8, the method further comprising, prior to the step of administration, the step of
diagnosing the subject as having a herpes virus infection.

18. The method of claim 17, wherein the subject is identified as testing negative for an HIV infection.

19. The method of claim 17, wherein the effective amount is sufficient to reduce viral replication by at least about 85% or more.

20. The method of claim 8, wherein the subject is identified as having an acyclovir-resistant herpes virus infection.

21. (canceled)

22. (canceled)

23. (canceled)

24. (canceled)

25. The method of claim 1, wherein the herpes virus is an alpaherpesvirus, a betaherpesvirus, or a gammaherpesvirus.

26. The method of claim 1, wherein the herpes virus is selected from the group consisting of Herpes simplex virus Type 1 (HSV-1), Herpes simplex virus Type 2 (HSV-2), Epstein Barr virus (EBV), Cytomegalovirus (CMV), Varicella Zoster Virus (VZV), Herpes lymphotropic virus, Human herpes virus 6 (HHV-6), Human herpes virus 7 (HHV-7), Human herpes virus 8 (HHV-8), and Kaposi's sarcoma-associated herpes virus (KSHV).

27. The method of claim 1, wherein the herpes virus is HSV-1 or HSV-2.

28. A pharmaceutical composition comprising an effective amount of an agent select from the group consisting of Raltegravir, 118-D-24, L-841411, elvitegrevir, MK-2048, XZ100, XZ99, XZ45, XZ15, XZ49, XZ48, and XZ50; or a derivative or analog thereof; or an agent that reduces the biological activity of a herpes virus polypeptide having functional and/or structural homology to a human immunodeficiency virus (HIV) integrase formulated for topical administration.

29. An immunogenic composition comprising an effective amount of an isolated herpes virus comprising an alteration in an ICP8 nucleic acid sequence, wherein the alteration decreases viral replication in a cell.

30. (canceled)

31. (canceled)

32. (canceled)

33. (canceled)

34. A method of inhibiting recombination mediated by ICP8 or an ICP8 homolog comprising contacting the ICP8 or ICP8 homolog with an agent selected from the group consisting of Raltegravir, 118-D-24, L-841411, elvitegrevir, MK-2048, XZ100, XZ99, XZ45, XZ15, XZ49, XZ48, and XZ50; or a derivative or analog thereof.

35. (canceled)

36. (canceled)
